(19) United States
(12) Patent Application Publication
(10) Pub. No.: US 2004/0058948 A1
(43) Pub. Date: Mar. 25, 2004

(54) Mappicine analogs, methods of inhibiting retroviral reverse transcriptase and methods of treating retroviruses

(76) Inventors: Dennis P. Curran, Pittsburgh, PA (US); Michael A. Parniak, Pittsburgh, PA (US); Ana Gabarda, New York, NY (US)

Correspondence Address: HENRY E. BARTONY, JR., LAW & FINANCE BUILDING, SUITE 1801, 429 FOURTH AVENUE, PITTSBURGH, PA, 15219, US

(21) Appl. No.: 10/378,467

(22) Filed: Mar. 3, 2003

Related U.S. Application Data
(60) Provisional application No. 60/361,023, filed on Mar. 1, 2002.

(51) Int. Cl. A61K 31/4745

(52) U.S. Cl. 514/285

(57) ABSTRACT

A method of inhibiting retroviral reverse transcriptase or hepadnaviral reverse transcriptase includes the step of administering a pharmaceutically effective amount of a mappicine analog or a pharmaceutically acceptable salt thereof. A method of treating a patient infected with a retrovirus or hepadnavirus includes the step of administering a pharmaceutically effective amount of a mappicine analog or a pharmaceutically acceptable salt thereof

### CROSS-REFERENCE TO RELATED APPLICATION

This application claims the benefit of U.S. Provisional Patent Application Serial No. 60/361,023, filed Mar. 1, 2002, the disclosure of which is incorporated herein by reference.

### GOVERNMENTAL INTERESTS

This invention was made with government support under grant GM33372 and grant GM33678 awarded by the National Institutes of Health. The government has certain rights in this invention.

### BACKGROUND OF THE INVENTION

The present invention relates to novel mappicine analogs, to methods of inhibiting retroviral reverse transcriptase and to methods of treating viruses, and, particularly, to novel mappicine analogs and the methods of treating retroviruses.

References set forth herein may facilitate understanding of the present invention or the background of the present invention. Inclusion of a reference herein, however, is not intended to and does not constitute an admission that the reference is available as prior art with respect to the present invention.

Certain 1H-pyrano[3′,4′:6,7]indolizino[1,2-b]quinolinones, such as camptothecins have been shown to have anticancer and antiviral activity. Indeed, a number of camptothecins are in use as anticancer agents. Although camptothecins possess antiviral activity, they exhibit certain characteristics that are undesirable for antiviral agents and have thus not been used as antiviral agents. For example, camptothecins inhibit mammalian topoisomerase I, inhibit host cell DNA replication, and are cytotoxic to mammalian cells.

It has been shown that substituted indolizino[1,2-b]quinolinones (and, in particular, mappicine ketones) that lack the α-hydroxylactone moiety of camptothecin are non-cytotoxic to mammalian cells, while exhibiting antiviral activity. In that regard, such substituted indolizino[1,2-b]quinolinones have been proposed for treating DNA viruses. See, for example, U.S. Pat. No. 5,883,255; Pendrak, I.; Barney, S.; Wittrock, R.; Lambert, D. M.; Kingsbury, W. D. “Synthesis and Anti-Hsv Activity of a-Ring-Deleted Mappicine Ketone Analog” J. Org. Chem. 1994, 59, 2623-2625; Pendrak, I.; Wittrock, R.; Kingsbury, W. D. “Synthesis and anti-HSV activity of methylenedioxy mappicine ketone analogs” J. Org. Chem. 1995, 60, 2912-2915.

A number of mappicine compounds are disclosed in de Frutos, O.; Curran, D. P. “Solution phase synthesis of libraries of polycyclic natural product analogues by cascade radical annulation: Synthesis of a 64-member library of mappicine analogues and a 48-member library of mappicine ketone analogues” J. Comb. Chem. 2000, 2, 639-649; Govindachari, T. R.; Ravindranath, K. R.; Viswanathan, N. “Isolation and Structure of Mappicine” J. Chem. Soc., Perkin 1 1974, 1215-1221; Ome Das, Biswanath; Madhusudhan, Purushotham. “Chemoenzymatic synthesis of (S)- and (R)-mappicines and their analogs,” Journal of Chemical Research, Synopses, 2000, 10, 476-477; Das, Biswanath; Madhusudhan, P., “Biochemical studies on natural products. V. Enantioselective synthesis of (S)- and (R)-mappicines and their analogues,” Tetrahedron, 1999, 55(25), 7875-7880; Allaudeen, Hameed Sheik; Berges, David Alan; Hertzberg, Robert Philip; Johnson, Randall Keith; Kingsbury, William Dennis; Petteway, Stephen Robert, Jr. “Preparation of substituted indolizino[1,2-b]quinolinones” Published PCT Int. Appl. WO 9207856 1992; Dodds, Helen M.; Craik, David J.; Rivory, Laurent P., “Photodegradation of Irinotecan (CPT-11) in Aqueous Solutions: Identification of Fluorescent Products and Influence of Solution Composition,” J. Pharm. Sci., 1997, 86(12), 1410-1416; Sawada, Seigo; Muraji, Ko., Preparation of camptothecin derivatives as antitumor agents, Jpn. Kokai Tokkyo Koho (1992); and Fortunak, J. M. D.; Mastrocola, A. R.; Mellinger, M.; Wood, J. L. “Preparation of mappicine ketones from camptothecins: Chemistry of the camptothecin E ring” Tetrahedron Lett. 1994, 35, 5763-5764, the disclosures of which are incorporated herein by reference.

Viruses are either DNA viruses or RNA viruses, but never both. DNA viruses can be divided into two groups: (1) those that have their genes on a double-stranded DNA molecule (dsDNA) (for example, smallpox); and (2) those that have their genes on a molecule of single-stranded DNA (ssDNA) (for example, Adeno-Associated Virus).

RNA viruses can be divided into four groups: (1) those with a genome that consists of single-stranded antisense RNA; that is, RNA that is the complement of the message sense (also called negative-stranded RNA; examples include measles and Ebola); (2) those with a genome that consists of single-stranded sense RNA; that is, the RNA has message sense (can act as a messenger RNA-mRNA) (also called positive-stranded RNA; for example, poliovirus); (3) those with a genome made of several pieces of double-stranded RNA (for example, reovirus) and (4) retroviruses, in which RNA (also single-stranded) is copied by reverse transcriptase into a DNA genome within the host cell (for example, human immunodeficiency virus (HIV))

Retroviral Reverse Transcriptase. Retroviruses carry their genetic information as RNA, but must replicate through a double-strand DNA intermediate. Thus, following recognition and entry into a susceptible cell, the retroviral genomic RNA must be converted into viral DNA. Multiple steps are involved in this crucial step of replication, each of which is catalyzed by the retroviral enzyme reverse transcriptase (RT). This enzyme is therefore multifunctional, and possesses three enzymatic activities, RNA-dependent DNA polymerase activity (RDDP), DNA-dependent DNA polymerase activity (DDDP), and ribonuclease H activity (RNase H).

Ribonuclease H (RNase H) is one of a family of enzymes termed nucleases, which act to hydrolyse nucleic acids. RNase H is unique among nucleases in that it selectively degrades the RNA component of an RNA/DNA duplex molecule, a double-strand nucleic acid comprised of one strand of ribonucleic acid (RNA) bound to a complementary strand of deoxyribonucleic acid (DNA) via Watson-Crick base pairing.

Ribonucleases H are ubiquitous, found in virtually all organisms, as well in several types of viruses, including retroviruses and hepadnavirus.

Ribonuclease H performs critical functions in the replication of several human pathogenic viruses, including retroviruses such as the human immunodeficiency virus (HIV) types 1 and 2, and the human T-cell leukemia viruses (HTLV) types 1 and 2. In addition, ribonuclease H is essential for the replication of the human hepadnavirus, hepatitis B virus (HBV).

Several retroviruses are human pathogens. These include the human immunodeficiency viruses type 1 and 2 (HIV-1 and HIV-2), and the human T-cell leukemia viruses types 1 and 2 (HTLV-1 and HTLV-2). Of these, HIV-1 is by far the most serious pathogen. HIV-1 infection leads to AIDS, an incurable and inevitably fatal disease. Since identification of the virus in the early 1980's, it is estimated that more than 58 million individuals have been infected with HIV-1, and of these nearly 25 million have died of AIDS. HIV-1 infection remains one of the most serious infectious disease problems worldwide.

A variety of biological agents are currently in use for the treatment of HIV-1 infections. HIV-1 RT has been, and remains, an important target for antiviral development. Many inhibitors of HIV-1 RT have been discovered, including nucleoside reverse transcriptase inhibitors (NRTI) such as 3′-azido-3′-deoxythymidine (AZT) and 2′,3′-dideoxy-3′-thiacytidine (3TC) and nonnucleoside reverse transcriptase inhibitors (NNRTI) such as nevirapine, delavirdine and efavirenz. However, virtually all inhibitors of HIV-1 RT are directed against the RDDP and/or DDDP activity of RT. Very few inhibitors of the ribonuclease H activity of HIV-1 (and HIV-2) reverse transcriptase have been described, and none are in clinical use.

Although current therapeutics are initially very effective at controlling the course of HIV spread in an infected individual, thereby improving the quality of life and longevity of HIV-infected patients, prolonged therapy inevitably leads to viral resistance to these drugs. Resistance to RT inhibitors correlates with mutations in RT, and resistance to protease inhibitors correlates with mutations in the HIV protease. Clinical appearance of drug-resistant HIV imparts an unfavorable prognosis. In addition, the transmission of drug-resistant HIV variants from an infected treated individual to a previously naive individual is becoming a serious problem. Drug therapies for use by these newly infected patients are restricted because of the infection by drug resistant virus.

There is therefore an urgent need to identify new inhibitors of HIV replication, especially inhibitors that act on new viral targets, not presently targeted by current chemotherapies. These new targets include the ribonuclease H activity associated with the viral reverse transcriptase. Identification of compounds that inhibit HIV-1 reverse transcriptase associated RNase H has been identified as a research priority by a number of organizations, including the United States National Institutes of Health (NIH).

Hepadnaviral Reverse Transcriptase. Human hepatitis B virus (HBV) is a major worldwide health threat and is responsible for the majority of the 1 to 2 million deaths annually from hepatitis. HBV is a member of the hepadnavirus family. Hepadnaviruses are small enveloped DNA viruses that replicate through an RNA intermediate. This replication mechanism therefore requires reverse transcription, to convert the RNA intermediate into viral DNA, a process carried out by the hepadnaviral P protein or reverse transcriptase. As is the case with retroviral reverse transcriptases, hepadnaviral P protein must be multifunctional to carry out reverse transcription. Thus, the protein possesses RNA-directed DNA polymerase and DNA-directed DNA polymerase activities, and ribonuclease H activity.

There are very few treatments available for HBV infection. These include interferon therapy or liver transplantation, both of which are expensive and at best only partially successful. Recently, the nucleoside analog 3TC has been approved for treatment of chronic infection and transplant patients. This nucleoside is directed against the DNA polymerase activity of the HBV DNA polymerase (hepadnaviral P protein). Additional therapies need to be developed. The hepadnaviral P protein-associated ribonuclease H provides a target for this development.

Objects of the present invention thus include development of reverse transcriptase inhibitors, development of RNase H inhibitors and development of improved methods of treatment of retroviruses, including HIV, and hepadnaviruses, including hepatitis B virus.

### SUMMARY OF THE INVENTION

In general, the inventors have discovered that analogs of the natural product mappicine or mappicine analogs inhibit retroviral reverse transcriptase and/or hepadnaviral reverse transcriptase by, for example, inhibiting the RNA-dependent DNA polymerase activity of reverse transcriptase and/or inhibiting the RNase H activity of reverse transcriptase (for example, HIV reverse transcriptase). Indeed, certain mappicine analogs were found to exhibit inhibition of the enzyme RNase H with a potency comparable to or better than the best currently known inhibitors. In that regard, the present invention provides in one aspect thereof a method of inhibiting retroviral reverse transcriptase in a patient (for example, a person or a mammal) infected with a retrovirus or hepadnavirus including the step of treating the patient with a pharmaceutically effective amount of a mappicine analog or a pharmaceutically acceptable salt thereof. The present invention also provides a method of treating a patient infected with a retrovirus or hepadnavirus with a pharmaceutically effective amount of a biologically active mappicine analog or a pharmaceutically acceptable salt thereof.

Examples of retroviral infections of humans that can be treated with the mappicine compounds of the present invention include the human immunodeficiency viruses HIV-1 and HIV-2 and human T-cell leukemia virus (HTLV-1 and HTLV-2). Treatable retroviral infections of nonhumans include, for example, feline immunodeficiency virus, feline leukemia virus (cats), bovine immunodeficiency virus, bovine leukemia virus (cattle), equine infectious anemia virus (horses), caprine arthritis-encephalitis virus (goats), and Rous sarcoma virus infection of chickens.

Examples of hepadnaviral infections of humans that can be treated with the mappicine compounds of the present invention include human hepatitis B virus (HBV).

As used herein, the term “mappicine analog” refers generally to a compound possessing the 11H-indolizino[1,2-b]quinolin-9-one ring skeleton. The analog can have substantially any organic substituent or functional group substituted in place of one or more of the hydrogen atoms on the ring skeleton. The analog can also have a maximum of one additional fused ring generated by replacing two hydrogens by a chain of atoms or groups selected from CH, CH₂, O, S, N, NH, N-alkyl or N-aryl. Preferred sizes of this additional ring are 5, 6, and 7.

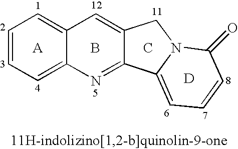

For example, mappicine analogs of the present invention can have the following general formulas:

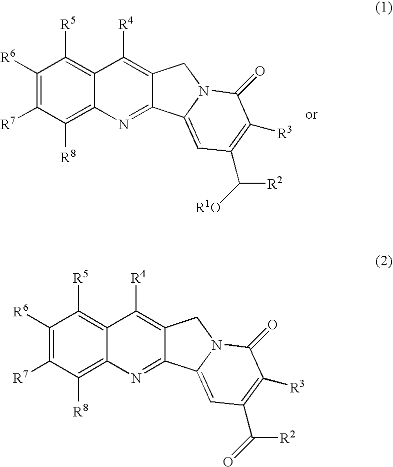

These compounds can also be represented with the following general formula:

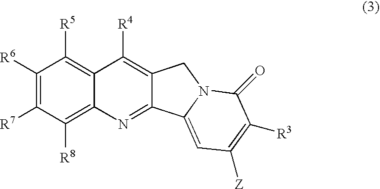

wherein Z is —CHOR¹R² or —C(O)R²;

wherein, R¹ is H, an alkyl group, an aryl group, —OC(O)OR^{a}, wherein R^{a} is an alkyl group, —C(O)R^{b} wherein R^{b} is an alkyl group, an aryl group, an alkoxy group, an amino group, an alkylamino group, a dialkylamino group, an aryl amino group, a diarylamino group, or an arylalkyl amino group;

R² is alkyl, aryl or arylalkyl;

R³ is H, alkyl, hydroxyalkyl or aryl;

R⁴, R⁵, R⁶, R⁷, and R⁸ are independently, the same or different, and are hydrogen, an alkyl group, an alkenyl group, an alkynyl group, an alkoxy group, an aryloxy group, an acyloxy group, a haloalkyl group, a perfluoroalkyl group, fluorine, chlorine, bromine, a carbamoyloxy group, a hydroxy group, a nitro group, a cyano group, a cyanoalkyl group, an azido group, an azidoalkyl group, a formyl group, a hydrazino group, a hydrazinoalkyl group, a hydroxyalkyl group, an alkoxyalkyl group, —NR^{l}R^{m}, wherein R^{l} and R^{m} are independently hydrogen, an alkyl group, an aryl group, an arylalkyl group, or —C(O)R, an alkylaminoalkyl group, a dialkylaminoalkyl group, an aryl aminoalkyl group, a diarylaminoalkyl group, an arylalkyl aminoalkyl group;

—OC(O)OR^{a}, wherein R^{a} is an alkyl group,

—C(O)R^{b} wherein R^{b} is an alkyl group, an aryl group, an alkoxy group, an amino group, an alkylamino group, a dialkylamino group, an aryl amino group, a diarylamino group, an arylalkyl amino group;

—SR^{c}, wherein R^{c} is hydrogen, —C(O)R^{b}, an alkyl group, or an aryl group; or

(CH₂)ₙSiR^{d}R^{c}R^{f} wherein n is an integer within the range of 0 through 10 and R^{d}, R^{e}and R^{f} are independently a C₁₋₁₀ alkyl group, a C₂₋₁₀ alkenyl group, a C₂₋₁₀ alkynyl group, an aryl group, a haloalkyl group, a cyanoalkyl group, an azidoalkyl group, a hydrazinoalkyl group, a hydroxyalkyl group, an alkoxyalkyl group, an aminoalkyl group, an alkylaminoalkyl group, a dialkylaminoalkyl group, an aryl aminoalkyl group, a diarylaminoalkyl group, an arylalkyl aminoalkyl group;

or wherein R⁴ and R⁵, R⁵ and R ⁶; R⁶ and R⁷; or R⁷ and R⁸ form together a chain of three or four groups selected from CH, CH₂, O, S, N, NH, N-alkyl or N-aryl.

However, The present invention also provides novel compounds of formula (3). In the event that some combination of substituents creates a chiral center or another form of an isomeric center in any compound of the present invention, all forms of such isomer(s) are considered to be aspects of the present inventions. When a compound of the present invention contains a chiral center, the present invention includes the racemic mixture, the pure enantiomers, and any enantiomerically enriched mixture thereof

The terms “alkyl”, “aryl” and other groups refer generally to both unsubstituted and substituted groups unless specified to the contrary. Unless otherwise specified, alkyl groups are hydrocarbon groups and are preferably C₁-C₁₅ (that is, having 1 to 15 carbon atoms) alkyl groups, and more preferably C₁-C₁₀ alkyl groups, and can be branched or unbranched, acyclic or cyclic. The above definition of an alkyl group and other definitions apply also when the group is a substituent on another group (for example, an alkyl group as a substituent of an alkylamino group or a dialkylamino group). The term “aryl” refers to phenyl or naphthyl. As used herein, the terms “halogen” or “halo” refer to fluoro, chloro, bromo and iodo.

The term “alkoxy” refers to —OR^{g}, wherein R^{g} is an alkyl group. The term “aryloxy” refers to —OR^{h}, wherein R^{h}, is an aryl group. The term acyl refers to —C(O)Rⁱ. The term “alkenyl” refers to a straight or branched chain hydrocarbon group with at least one double bond, preferably with 2-15 carbon atoms, and more preferably with 2-10 carbon atoms (for example, —CH═CHR^{j} or —CH₂CH═CHR^{j}). The term “alkynyl” refers to a straight or branched chain hydrocarbon group with at least one triple bond, preferably with 2-15 carbon atoms, and more preferably with 2-10 carbon atoms (for example, —C≡CR^{k} or —CH₂—C≡CR^{k}). The terms “alkylene,” “alkenylene” and “alkynylene” refer to bivalent forms of alkyl, alkenyl and alkynyl groups, respectively.

The groups set forth above, can be substituted with a wide variety of substituents to synthesize mappicine analogs retaining activity. For example, alkyl groups may preferably be substituted with a group or groups including, but not limited to, a benzyl group, a phenyl group, an alkoxy group, a hydroxy group, an amino group (including, for example, free amino groups, alkylamino, dialkylamino groups and arylamino groups), an alkenyl group, an alkynyl group, a halogen (for example, perfluoroalkyl) and an acyloxy group. In the case of amino groups (—NR^{l}R^{m}), R^{l} and R^{m} are preferably independently hydrogen, an acyl group, an alkyl group, or an aryl group. Acyl groups may preferably be substituted with (that is, Rⁱ is) an alkyl group, a haloalkyl group (for example, a perfluoroalkyl group), an alkoxy group, an amino group and a hydroxy group. Alkynyl groups and alkenyl groups may preferably be substituted with (that is, R^{j} and R^{k} are preferably) a group or groups including, but not limited to, an alkyl group, an alkoxyalkyl group, an amino alkyl group and a benzyl group.

The term “acyloxy” as used herein refers to the group —OC(O)R^{g}.

The term “alkoxycarbonyloxy” as used herein refers to the group —OC(O)OR^{g}.

The term “carbamoyloxy” as used herein refers to the group —OC(O)NR^{l}R^{m}.

For purpose of, for example, effecting separation of mappicine analogs prepared as a library in a combinatorial or parallel synthesis, R^{l} can also be a or fluorous tag. As used herein, the terms “fluorous tagging” or “fluorous-tagged” refers generally to attaching a fluorous moiety or group (referred to as a “fluorous tagging moiety,” a “fluorous tagging group” or simply a “fluorous tag”) to a compound to create a “fluorous-tagged compound”. Preferably, the fluorous tagging moiety is attached via covalent bond. However, other effective attachments such as ionic bonding, chelation or complexation can also be used. Fluorous tagging moieties facilitate separation of fluorous tagged compounds from other compounds as a result of differences in the fluorous nature of the compounds. Especially useful are fluorous separation methods such as fluorous liquid-liquid extraction, fluorous solid-liquid extraction, and/or fluorous chromatography.

As used herein, the term “fluorous”, when used in connection with an organic (carbon-containing) molecule, moiety or group, refers generally to an organic molecule, moiety or group having a domain or a portion thereof rich in carbon-fluorine bonds (for example, fluorocarbons, fluorohydrocarbons, fluorinated ethers and fluorinated amines). The terms “fluorous-tagged reagent” or “fluorous reagent,” thus refer generally to a reagent comprising a portion rich in carbon-fluorine bonds. As used herein, the term “perfluorocarbons” refers generally to organic compounds in which all hydrogen atoms bonded to carbon atoms have been replaced by fluorine atoms. The terms “fluorohydrocarbons” and “hydrofluorocarbons” include organic compounds in which at least one hydrogen atom bonded to a carbon atom has been replaced by a fluorine atom. Fluorous moieties and/or the attachment of fluorous moieties or tags to organic compounds are discussed for example, in U.S. Pat. Nos. 5,859,247, 5,777,121, and U.S. patent application Ser. Nos. 09/506,779, 09/565,087, 09/602,105, 09/952,188 and 09/877,944, the disclosures of which are incorporated herein by reference. Fluorous mixture synthesis of mappicines is discussed in Luo, Z. et al., “Fluorous Mixture Synthesis: A Fluorous-Tagging Strategy for the Synthesis of Separation of Mixtures of Organic Compounds,” Science, 2001, 291, 1766-1769, a copy of which is attached hereto and the disclosure of which is incorporated herein by reference.

Fluorous tags suitable for use in the present invention include, for example, a perfluoroalkyl group, a hydrofluoroalkyl group, a fluorinated ether group or a fluorinated amine group.

Perfluoroalkyl groups are preferably of 2 to 20 carbons. Hydrofluoroalkyl groups are preferably of 2 to 20 carbons and include up to one hydrogen atom for each two fluorine atoms. For example, perfluorinated ether groups can have the general formula —[(CF₂)ₓO(CF₂)_{y}]_{z}CF₃, wherein x, y and z are integers. Perfluorinated amine groups can, for example, have the general formula —[(CF₂)_{x′}(NR^{a})CF₂)_{y′}]_{z′}CF₃, wherein x′, y′ and z′ are integers and wherein R^{a} can, for example, be CF₃ or (CF₂)_{n′}CF₃, wherein n′ is an integer. Fluorinated ether groups and fluorinated amine groups suitable for use in the present invention need not be perfluorinated, however. Fluorinated ether groups are preferably of 3 to 20 carbons. Fluorinated amine groups are preferably of 4 to 20 carbons.

Certain groups such as hydroxy groups, amino groups and/or other groups of certain compounds of the present invention and certain compounds used in the methods of the present invention can be protected using protective groups as known in the art. Such protective groups include, but are not limited to, —SiR¹⁰R¹¹R¹² wherein R¹⁰, R¹¹, and R¹² are independently the same or different an alkyl group (preferably a lower alkyl group) or an aryl group; CHR^{x}OR^{y} where R^{x} is H or alkyl (preferably lower alkyl, and more preferably methyl) and R^{y} is alkyl (preferably lower alkyl) or CH₂C₆H₃RⁿR^{o} wherein Rⁿ and R^{o} are independently the same or different, ortho, meta or para H, alkyl (preferably lower alkyl), alkoxy, nitro, cyano, halo, phenyl, trifluoromethyl or azido; CH₂CH₂OR¹³ where R¹³ is alkyl, CH₂CH₂SiR¹⁰R¹¹R¹² or CH₂CCl₃; 2-tetrahydropyranyl; 4-methoxy-2-tetrahydropyranyl; 2-tetrahydrofuranyl; CH₂SR^{p} where R^{P} is alkyl (preferably lower alkyl); CH₂CH₂Si R¹⁰R¹¹R¹²; a tert-butyl group; CH₂C₆H₃R^{q}R^{r} wherein R^{q} and R^{r} are independently the same or different, ortho, meta, or para H, alkyl (preferably lower alkyl), alkoxy, nitro, cyano, halo, phenyl, trifluoromethyl or azido; or —C(O)R¹⁴ wherein R¹⁴ is H, alkyl (preferably lower alkyl), haloalkyl, aryl, alkoxy or OCH₂C₆H₃R^{s}R^{t}, wherein R^{s} and R^{t} are independently the same or different, ortho, meta, or para H, alkyl (preferably lower alkyl), alkoxy, nitro, cyano, halo, phenyl, trifluoromethyl or azido. Other suitable protecting groups as known to those skilled in the art are disclosed, for example, in Greene, T., Wuts, P. G. M., Protective Groups in Organic Synthesis, Wiley (1991) and other general references set forth below, the disclosures of which is incorporated herein by reference.

The protecting groups may be present in any precursors and intermediates and should protect the functional groups concerned against unwanted secondary reactions, such as acylations, etherifications, esterifications, oxidations, solvolysis, and similar reactions. In certain cases, the protecting groups may, in addition to this protection, effect a selective course of reactions. It is a characteristic of protecting groups that they lend themselves readily, i.e. without undesired secondary reactions, to removal, typically by solvolysis, reduction, photolysis or also by enzyme activity, for example under conditions analogous to physiological conditions, and that they are not present in the end-products. The specialist knows, or can easily establish, which protecting groups are suitable with the reactions mentioned hereinabove and hereinafter.

The protection of functional groups by protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene, “Protective Groups in Organic Synthesis”, Wiley, New York 1981, in “The Peptides”; Volume 3 (editors: E. Gross and J. Meienhofer), Academic Press, London and New York 1981, in “Methoden der organischen Chemie” (Methods of organic chemistry), Houben Weyl, 4th edition, Volume 15/1, Georg Thieme Vedag, Stuttgart 1974, in H. -D. Jakubke and H. Jescheit, “Aminosauren, Peptide, Proteine” (Amino adds, peptides, proteins), Verlag Chemie, Weinheim, Deerfield Beach, and Basel 1982, and in Jochen Lehmann, “Chemie der Kohlenhydrate: Monosaccharide und Derivate” (Chemistry of carbohydrates: monosaccharides and derivatives), Georg Thieme Verlag, Stuttgart 1974.

For purpose of biological activity, R¹, R², R³, R⁶, R⁷ and R⁸ are, in general, preferably not excessively bulky to maintain the activity of the resultant mappicine analog. Preferably, therefore, R¹, R², R³, R⁶, R⁷ and R⁸ independently have a molecular weight less than approximately 350. More preferably R¹, R², R³, R⁶, R⁷ and R⁸ independently have a molecular weight less than approximately 250. In general, the total molecular weight of the sum of all R¹, R², R³, R⁶, R⁷ and R⁸ groups preferably does not exceed about 750, and more preferably does not exceed about 600. Certain intermediates, such as fluorous tagged mappicine compounds of the present invention need not satisfy the above criteria.

Some of the mappicine analogs of the present invention can be prepared for pharmaceutical use as salts with inorganic acids such as, but not limited to, hydrochloride, hydrobromide, sulfate, phosphate, and nitrate. The mappicine analogs can also be prepared as salts with organic acids such as, but not limited to, acetate, tartrate, fumarate, succinate, citrate, methanesulfonate, p-toluenesulfonate, and stearate. Other acids can be used as intermediates in the preparation of the compounds of the present invention and their pharmaceutically acceptable salts. Likewise, for some analogs of the present invention, salts with organic (for example, amine) and inorganic (for example, sodium and potassium) bases can also be prepared.

The compounds of the present invention, for example, be administered by any conventional route of administration, including, but not limited to, intravenously, intramuscularly, orally, subcutaneously, intratumorally, intradermally, and parenterally. The pharmaceutically effective amount or dosage is preferably between 0.01 to 250 mg of one of the compounds of the present invention per kg of body weight. More preferably, the pharmaceutically effective amount or dosage is preferably between 0.1 to 40 mg of one of the compounds of the present invention per kg of body weight. In general, a pharmaceutically effective amount or dosage contains an amount of one of the compounds of the present invention effective to display antiretroviral behavior. Pharmaceutical compositions containing as an active ingredient one of the compounds of the present invention or a pharmaceutically acceptable salt thereof in association with a pharmaceutically acceptable carrier or diluent are also within the scope of the present invention.

The present invention also provides a pharmaceutical composition comprising any of the compounds of the present invention and a pharmaceutically acceptable carrier. The composition may, for example, contain between 0.1 mg and 3 g, and preferably between approximately 0.1 mg and 500 mg of the compounds of the present invention, and may be constituted into any form suitable for the mode of administration.

### BRIEF DESCRIPTION OF THE DRAWINGS

FIG. 1A illustrates synthetic schemes for the synthesis of mappicine analogs.

FIG. 1B illustrates a synthetic scheme for synthesis of a series of mappicine analogs of the present invention.

FIGS. 2A-2M illustrates chemical structures of a number of mappicine analogs of the present invention.

FIG. 3 illustrates a dose-response curve for compound AG 2M.

FIG. 4 illustrates a dose-response curve for compound AG 6M.

### DETAILED DESCRIPTION OF THE INVENTION

Recently, a fluorescence-based assay to perform preliminary screens in search of HIV RNase H inhibitors has been developed. The assay, which is carried out in 96-well microplates and is adaptable to robotics, is the first high-throughput screen for RNase H and is described in further detail in the Experimental section. HIV-1 RT and human RNase H were cloned, and thus comparative analysis of inhibitor action could be conducted simultaneously.

In the course of the present studies, it was speculated that elongated “flat” structures may be desirable for binding to RNase H domain. For example, the compound N-4-(t-butylbenzoyl)-2-hydroxy-1-napthaldehyde hydrazone (or more conveniently BBNH) is one of the most active RNase H inhibiting compounds discovered to date with an IC₅₀≈2 μm (wherein IC₅₀ refers to the Inhibitory Concentration that provides 50% reduction in target activity). See, for example, Borkow, G. et al., “Inhibition of the Ribonuclease H and DNA Polymerase Activities of HIV-1 Reverse Transcriptase by N-(4-tert-Butylbenzoyl)-2-hydroxy-1-naphthaldehyde Hydrazone,” Biochemistry 1997, 36, 3179-3185. Mappicine analogs similarly exhibit an elongated flat structure.

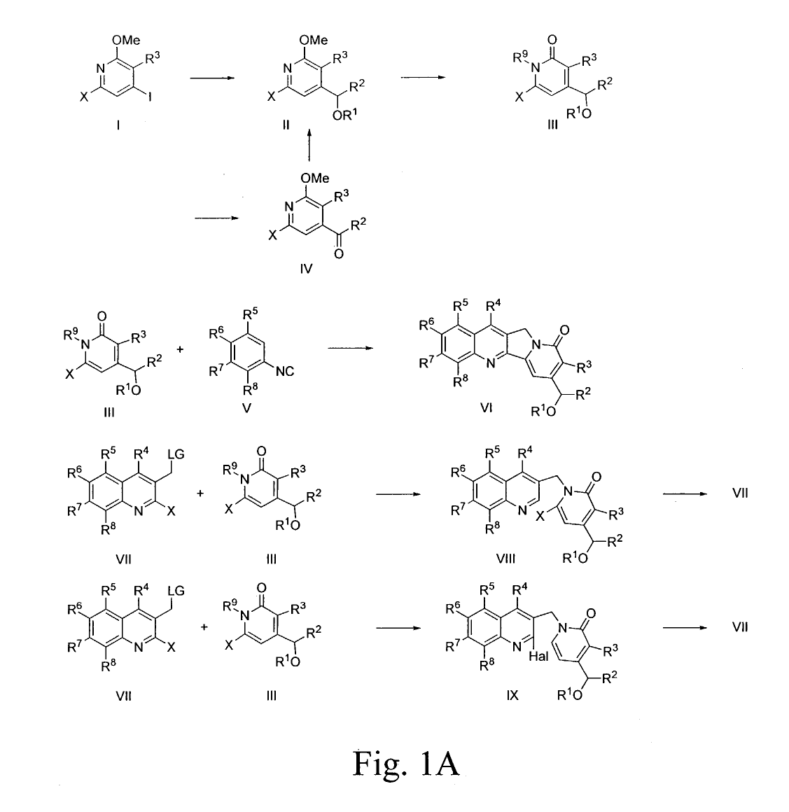

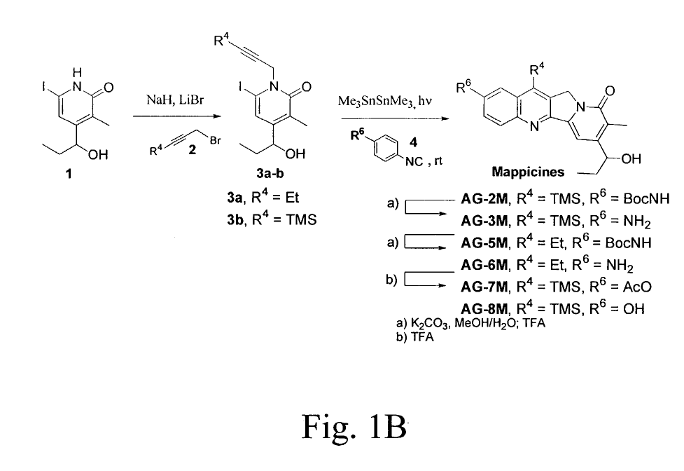

There are many ways to make camptothecin and mappicine analogs and substantially any of these can be used to make the compounds of the present invention. Several representative examples of preferred synthetic routes to make the compounds of this invention are summarized below and in FIG. 1A. Compounds of the general formula I with X and R³ as described above can be subjected to iodine/metal exchange and the resulting organometallic species (for example, a lithium or Grignard reagent) is contact with an aldehyde R²CHO to give II with R¹=H. Conversion of this compound to the other R¹ groups of this invention uses standard reactions. Also, I can be acylated, for example by Stille reaction with R²COSnBu₃, to give IV, which can be used for onward reactions in a manner substantially similar to II or converted to II by standard reduction. For certain sequences, the conversion of II, X=TMS to II, X=I can be useful and can be accomplished by iododesilylation with, for example, ICl. Demethylation of II, for example with TMSI or HI, followed by alkylation with R⁴CCCH₂Br, R⁴CH═CHCH₂Br or related allylating or propargylating agents gives III, R⁹=CH₂CCR⁴ or CH₂CH═CHR⁴. See, for example, Liu, H.; Ko, S. B.; Josien, H.; Curran, D. P. Tetrahedron Lett. 1995, 36, 8917-8920.

Compounds III can be used in many ways to make the mappicines of the present invention. For example, reaction of III, R⁹═CH₂CCR⁴ or CH₂CH═CHR⁴ and X=a radical precursor with isonitrile V with R⁵-R⁸ as described above under conditions for cascade radical annulation (see FIG. 1A) provides mappicines VI. Preferred radical precursors are iodine and bromine. Reaction of III, X=Br or I, with V to give VI can also be promoted by certain transition metals such as, for example, salts or complexes of palladium. See, for example, U.S. Patent Provisional Patent Application Serial No. 60/382,292, the disclosure of which is incorporated herein by reference. Reaction of III, R⁹=H and X=chlorine, bromine, or iodine with VII, X=H and LG=a leaving group under conditions for N-alkylation provides VIII, which can in turn can be converted to VII under either radical conditions (for example, treatment with Bu₃SnH) or organometallic conditions, for example, treatment with palladium catalysts. Likewise, reaction of III, R⁹=H and X=H with VII, LG=leaving group and X=chlorine, bromine or iodine as above provides IX, which again can be converted to VII under radical or organometallic conditions. See, for example, Comins, D. L.; Hong, H.; Jianhua, G. Tetrahedron Lett. 1994, 35, 5331-5334. Comins, D. L.; Hong, H.; Saha, J. K.; Gao, J. H. J. Org. Chem. 1994, 59, 5120-5121. Comins, D. L.; Saha, J. K. J. Org. Chem. 1996, 61, 9623-9624.

Mappicines VII can be converted to mappicine ketones by using standard alcohol oxidations. In turn, if ketones such are IV are used in the synthetic sequence, mappicine ketones result directly, and these can be converted to mappicines by standard reductions.

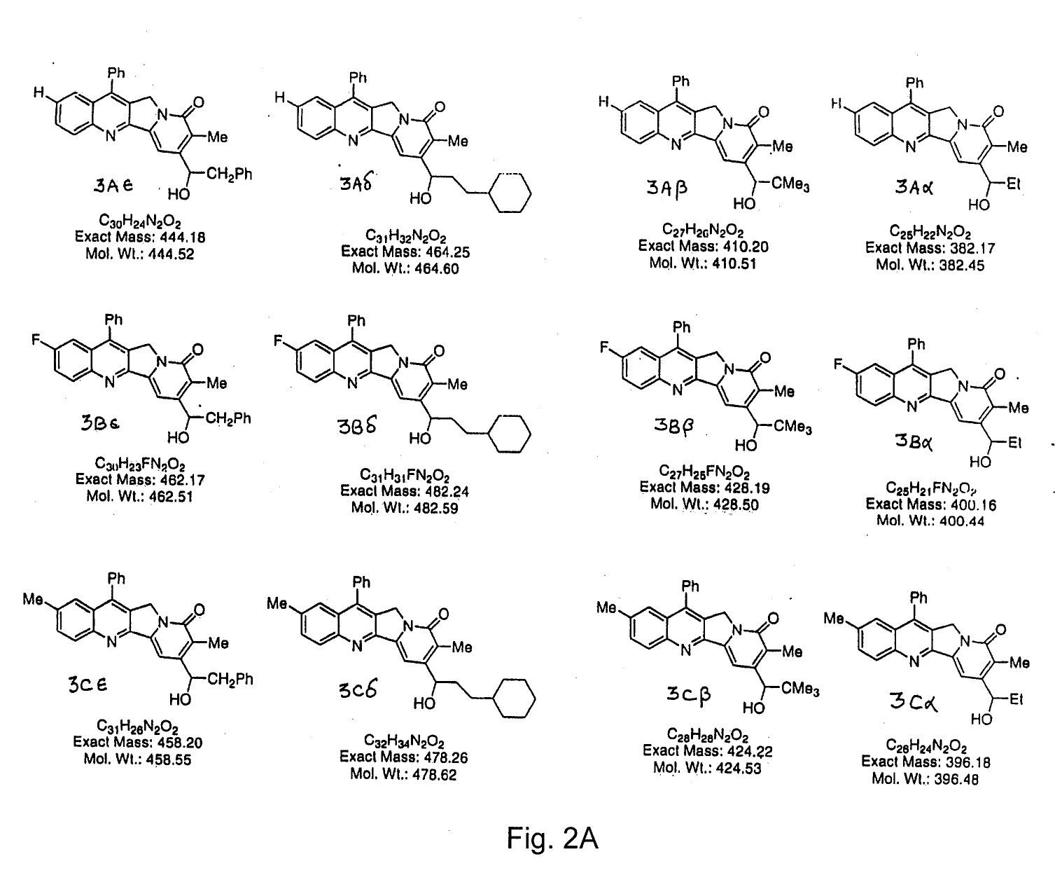

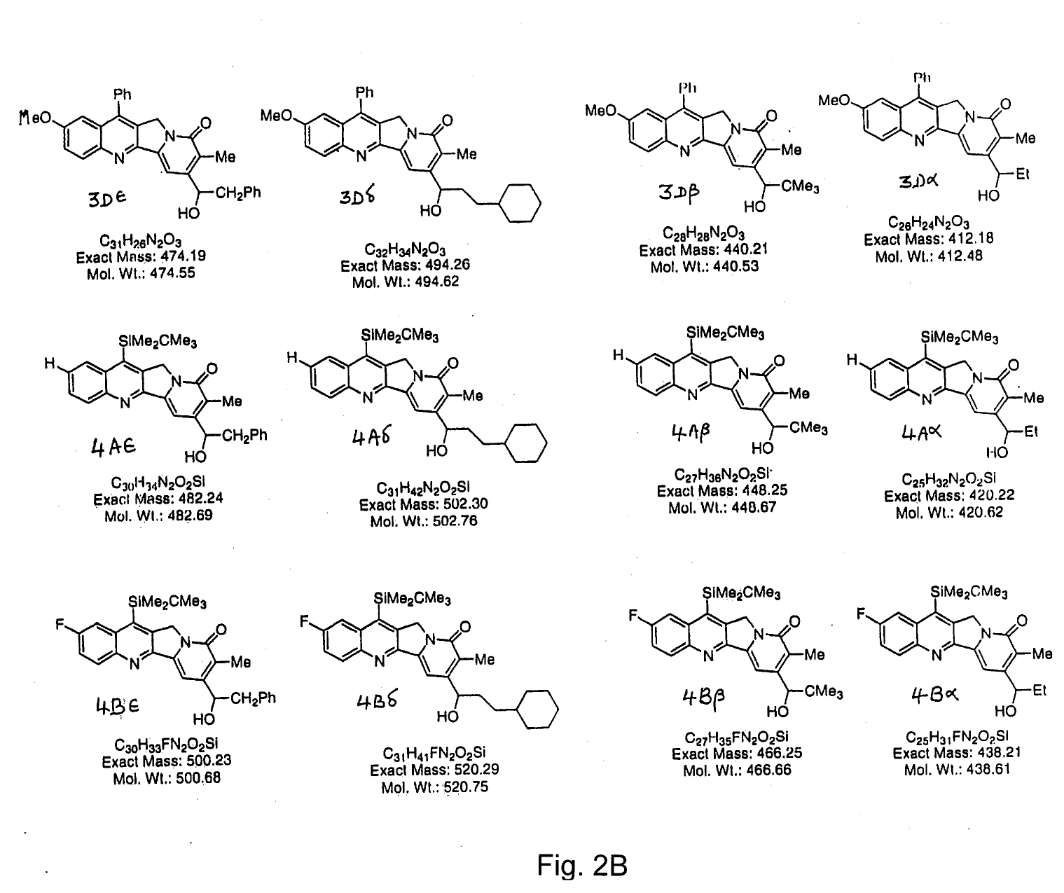

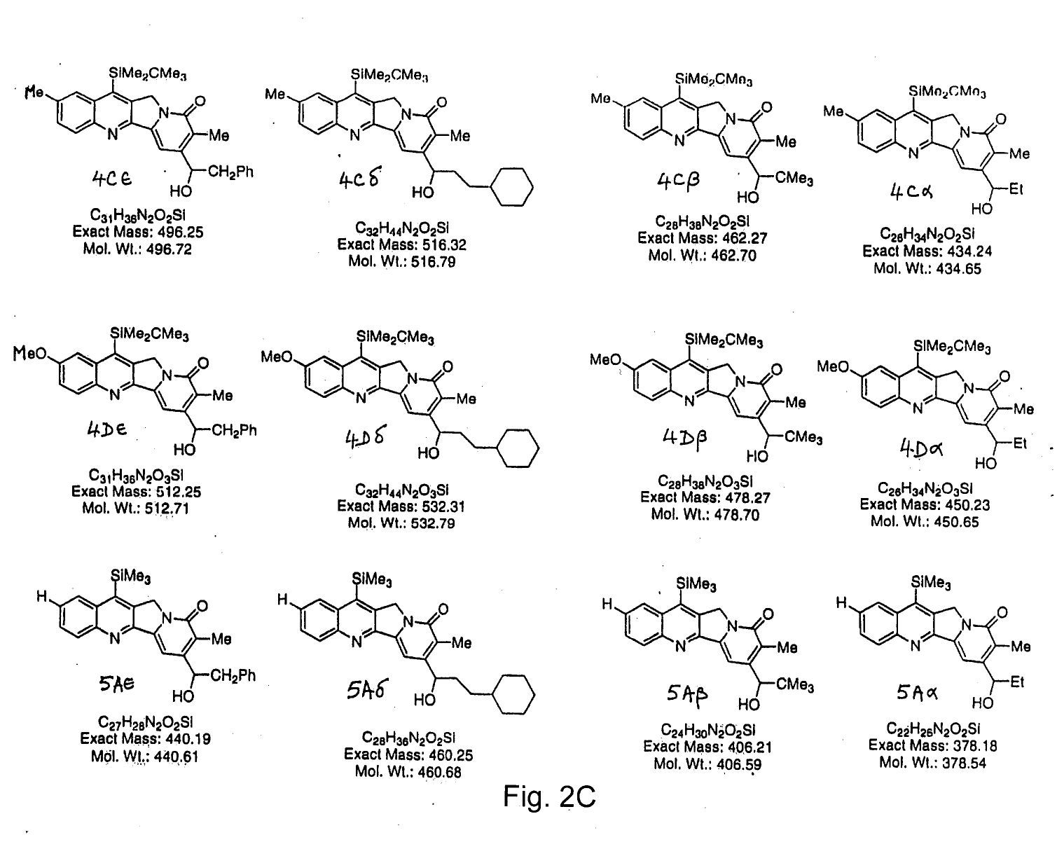

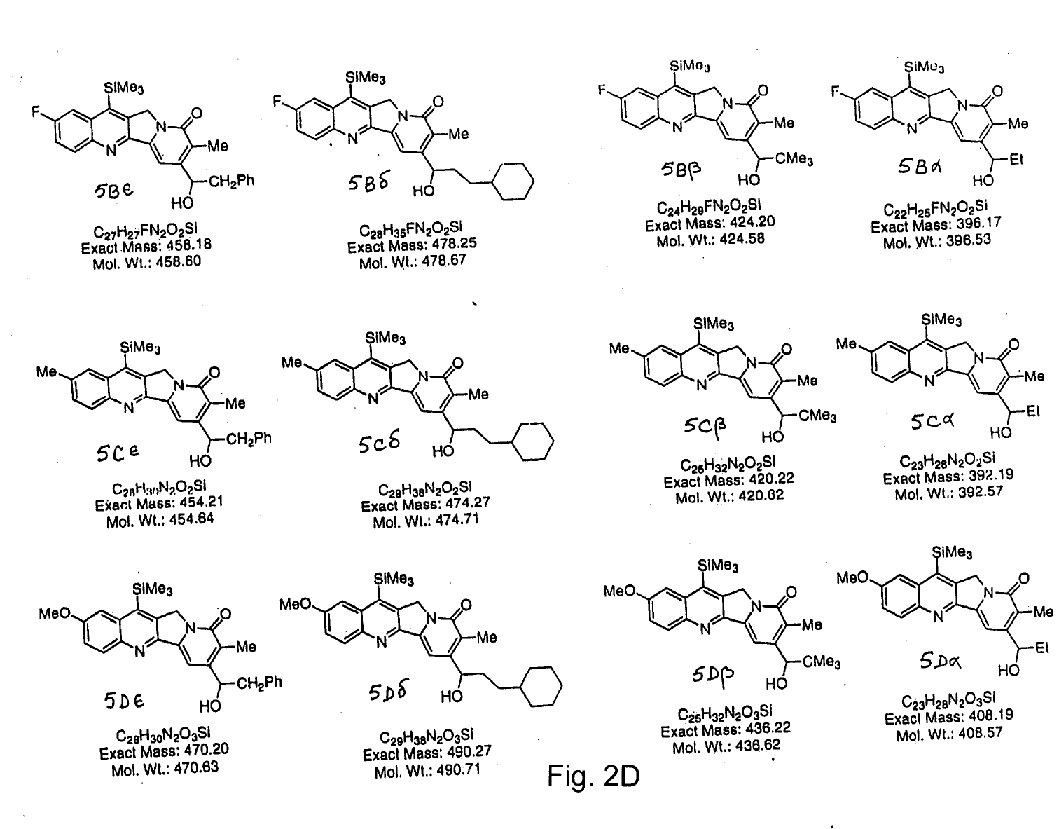

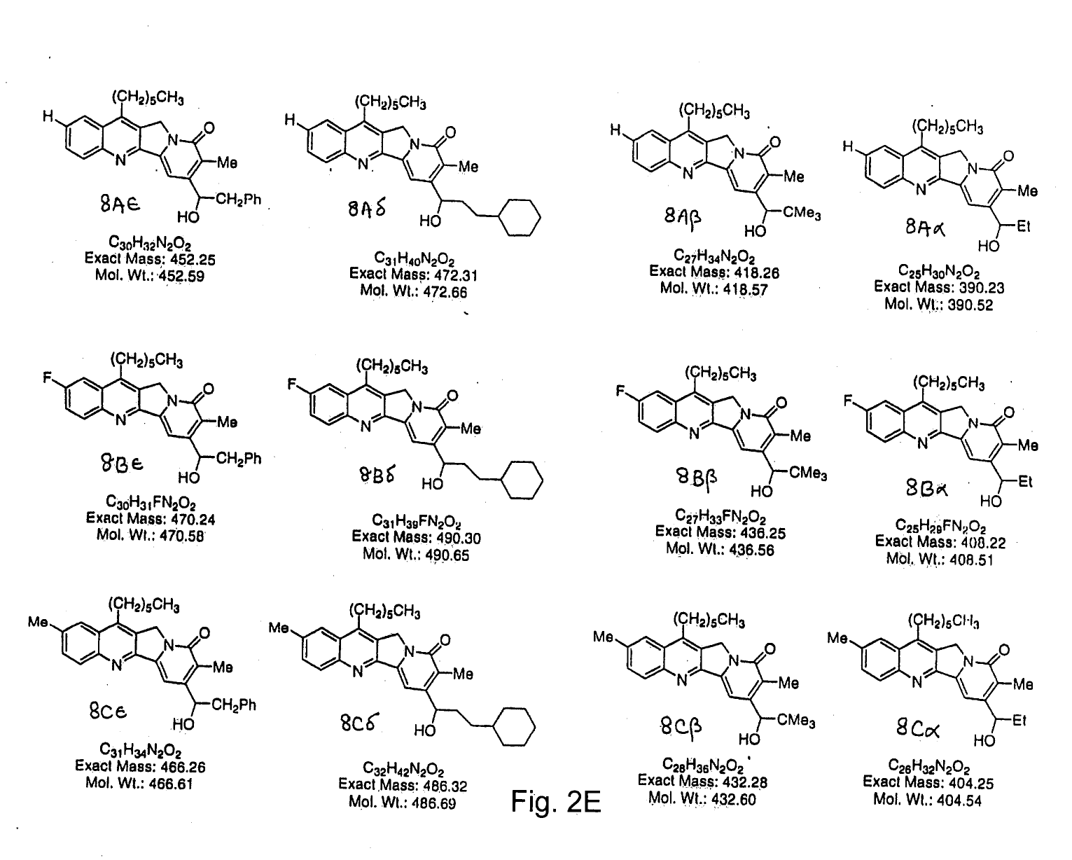

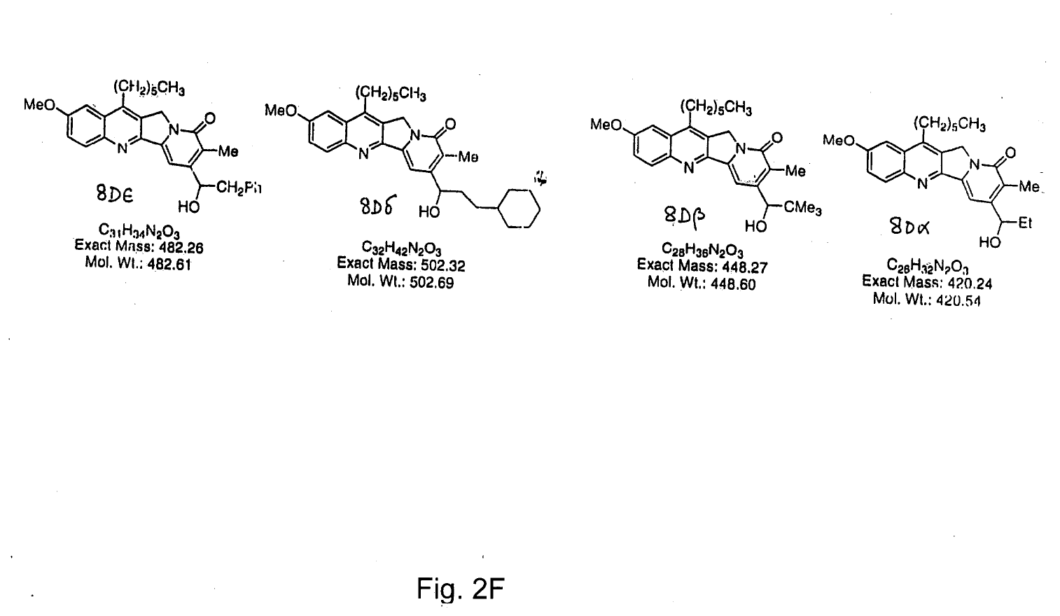

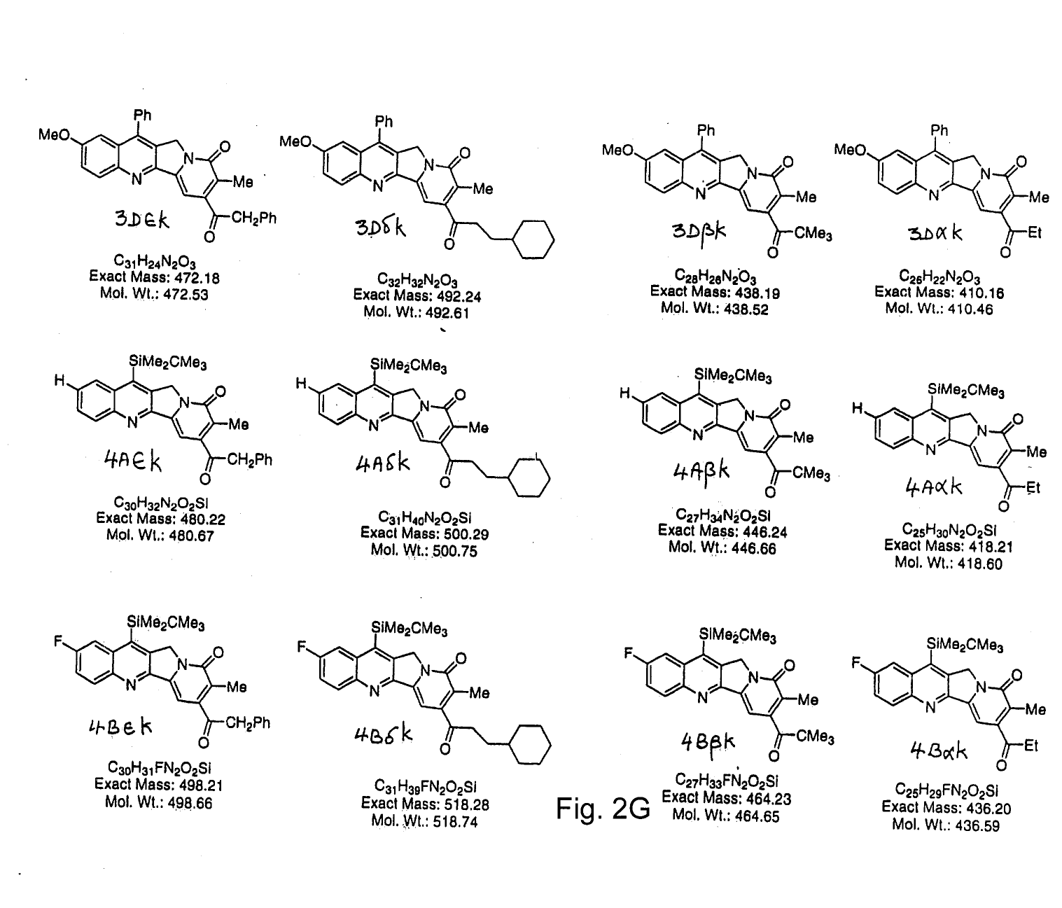

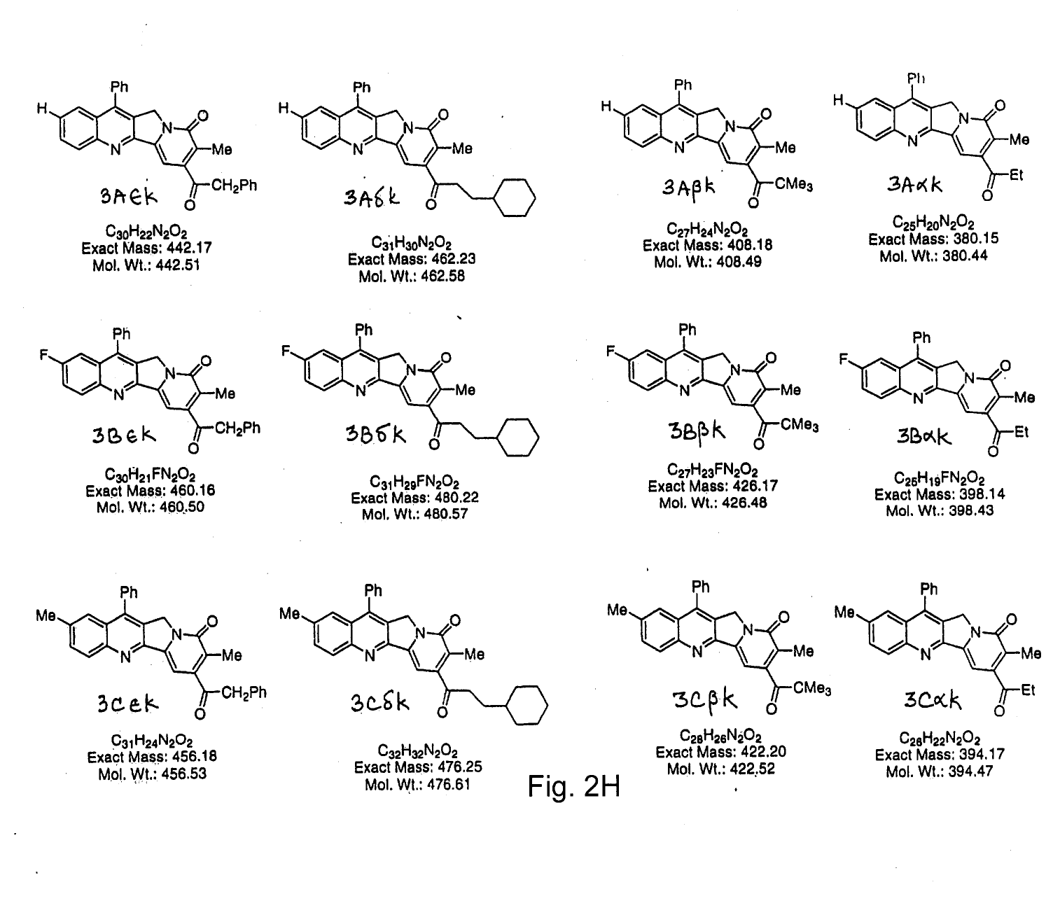

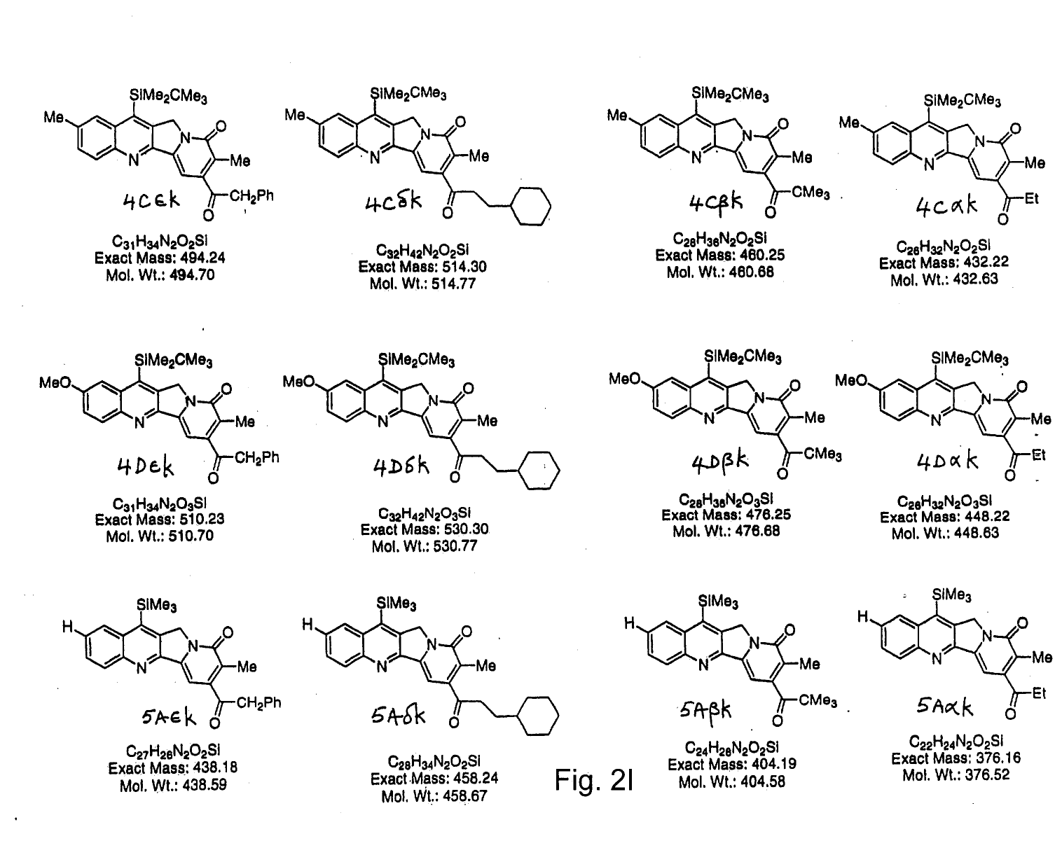

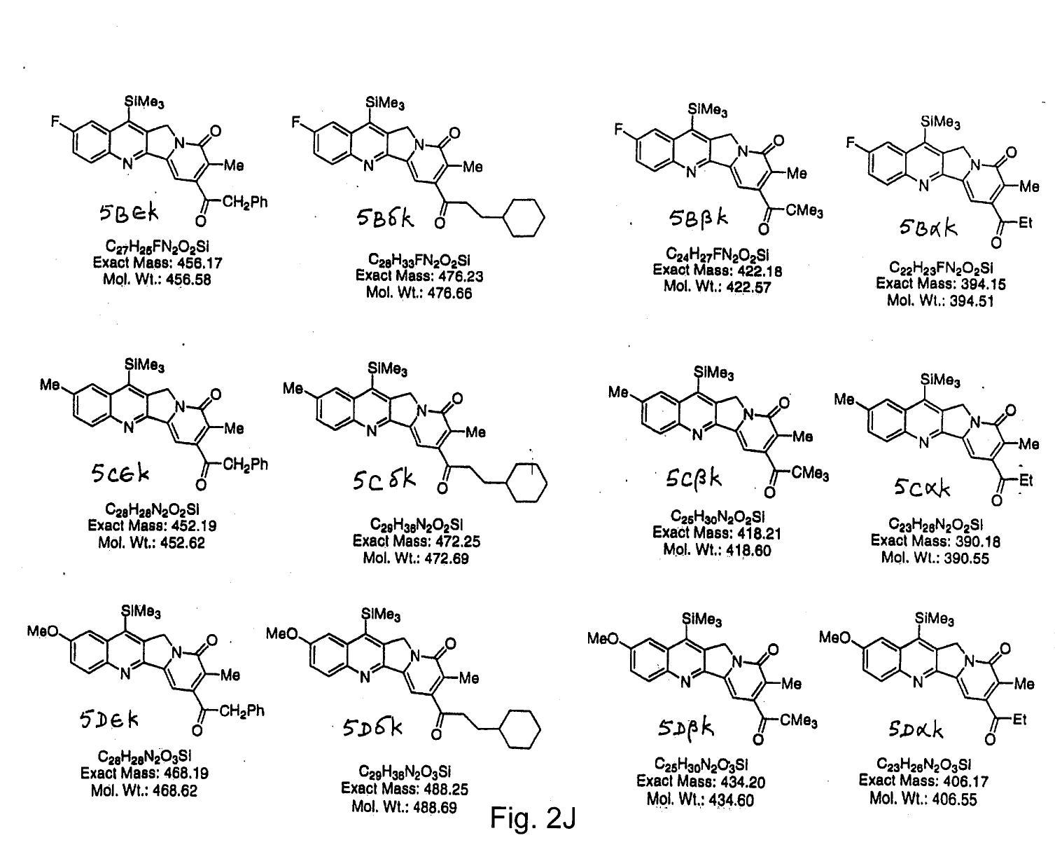

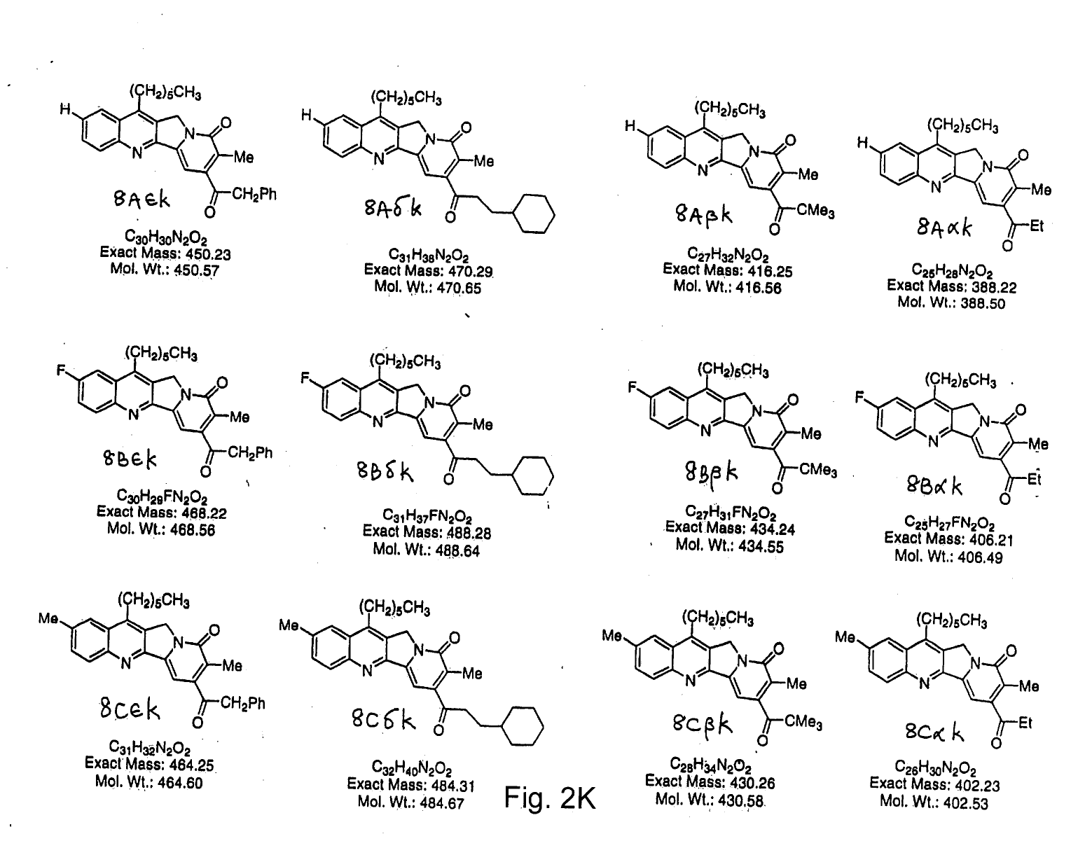

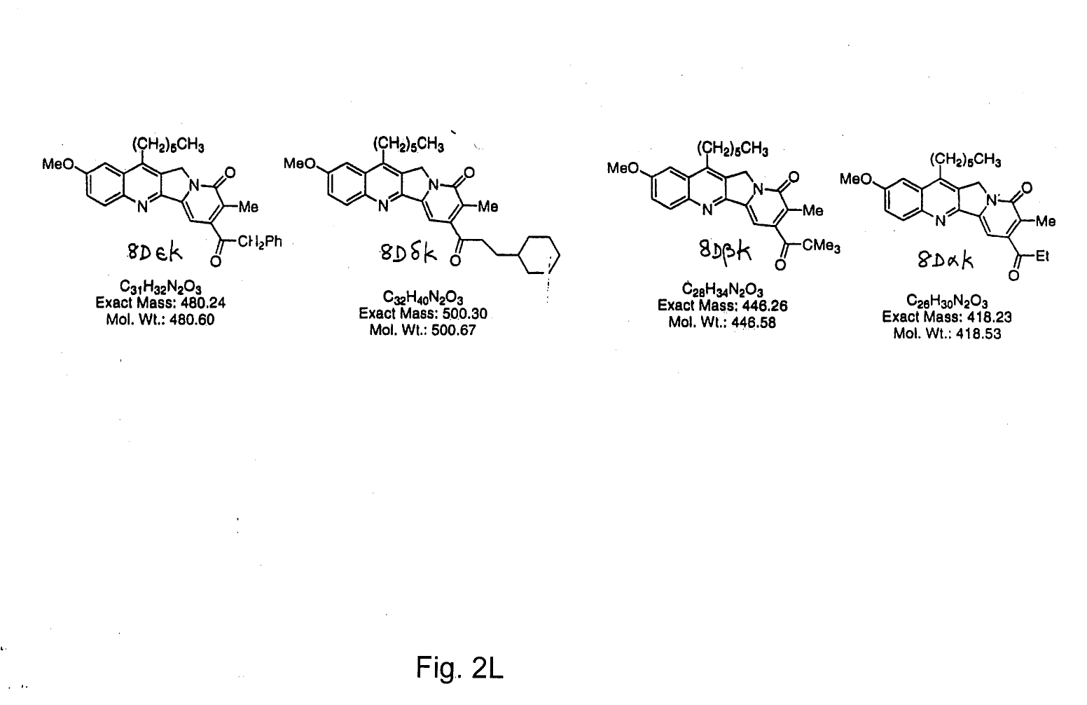

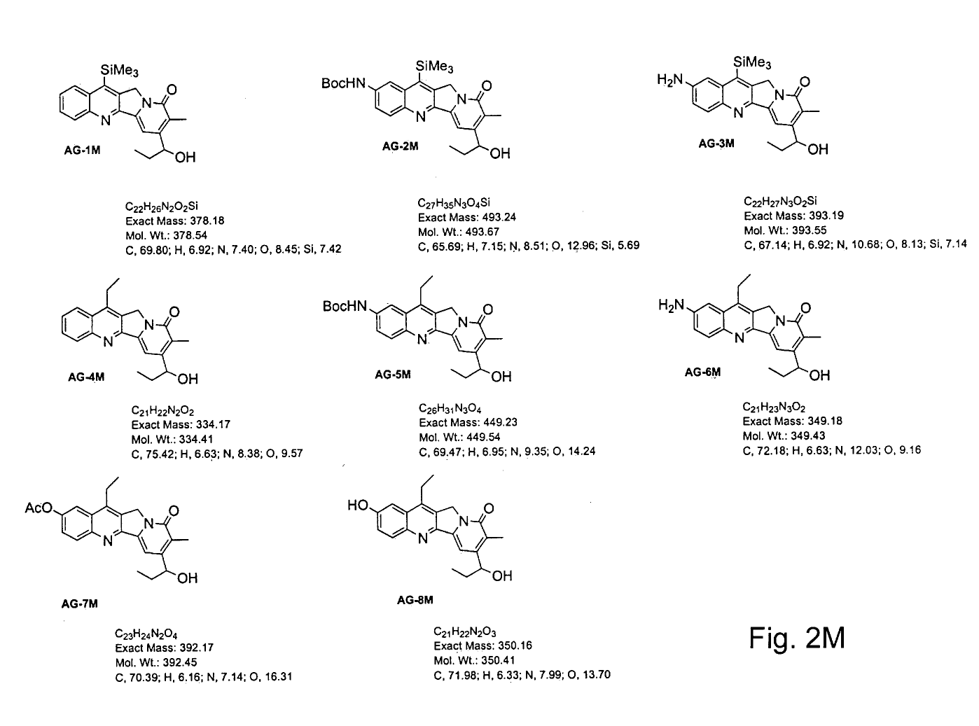

Libraries of mappicine analogs were studied in an HIV RNase H assay (see FIG. 1B). Mappicine analogs of the present invention (see, for example, FIGS. 2A through 2M) can, for example, be prepared via a parallel library synthesis via a cascade radical annulation method as disclosed in de Frutos, O.; Curran, D. P. J. Comb. Chem. 2000, 2, 639, the disclosure of which is incorporated by reference. Mappicine analogs can also be prepared in a traditional (non-parallel) fashion as described below. In several of the representative non-parallel syntheses described below, 12- and/or 2-substituted (for example, 2-hydroxy, 2-AcO, 10-BocNH or 2-amino) mappicine analogs were prepared using the synthetic scheme set forth below. Substitutions at corresponding positions in camptothecin analogs are, for example, known to enhance biological activity in certain camptothecin analogs.

In the synthesis, intermediate 1 was prepared according to the reported procedure. de Frutos, O.; Curran, D. P., J. Comb. Chem., 2000, 2, 639. Beginning with 1, the synthesis of, for example, 2-hydroxy and 2-amino mappicine analogs followed the sequence set forth below. As set forth in FIG. 1B, first, N-alkylation of iodopyridone 1 with the corresponding propargyl bromide 2 (R⁴ is, for example, Et or TMS) provided the radical precursors 3a and 3b. Second, [4+1] cascade radical reaction of pyridones 3a/b with the corresponding isonitriles 4 (R⁶ is, for example BocNH or AcO) gave rise to mappicine analogs AG-2M, AG-5M and AG-7M. Subsequently, 2-AcO and 2-BocNH deprotection yielded 2-hydroxy and 2-amino mappicine analogs AG-3M, AG-6M and AG-8M.

Assay results for a number of mappicine analogs illustrated in FIGS. 2A through 2M are set forth in Table 1 below. The designation set forth in FIGS. 2A through 2M for the corresponding chemical structures are used throughout this specification. The assay results provided in Table 1 were obtained at 10 μM inhibitor concentrations.

TABLE 1

Residual activity (%)

| Compounds | Polymerase | RNase H |
|---|---|---|
| 3Aε | 34 | 99 |
| 3Aδ | 32 | 104 |
| 3Aβ | 54 | 95 |
| 3Aα | 90 | 97 |
| 3Bε | 28 | 91 |
| 3Bδ | 81 | 99 |
| 3Bβ | 78 | 75 |
| 3Bα | 54 | 83 |
| 3Cε | 76 | 101 |
| 3Cδ | 74 | 93 |
| 3Cβ | 70 | 84 |
| 3Cα | 64 | 96 |
| 3Dε | 61 | 79 |
| 3Dδ | 60 | 67 |
| 3Dβ | 85 | 70 |
| 3Dα | 69 | 78 |
| 4Aε | 83 | 93 |
| 4Aδ | 45 | 95 |
| 4Aβ | 71 | 101 |
| 4Aα | 67 | 104 |
| 4Bε | 61 | 89 |
| 4Bδ | 44 | 90 |
| 4Bβ | 49 | 113 |
| 4Bα | 98 | 108 |
| 4Cε | | |
| 4Cδ | 61 | 109 |
| 4Cβ | 67 | 94 |
| 4Cα | 94 | 91 |
| 4Dε | 49 | 76 |
| 4Dδ | 51 | 87 |
| 4Dβ | 47 | 91 |
| 4Dα | 77 | 79 |
| 5Aε | 54 | 93 |
| 5Aδ | 83 | 78 |
| 5Aβ | 103 | 77 |
| 5Aα | 32 | 93 |
| 5Bε | 49 | 88 |
| 5Bδ | 78 | 103 |
| 5Bβ | 74 | 102 |
| 5Bα | 82 | 105 |
| 5Cε | 87 | 93 |
| 5Cδ | 41 | 103 |
| 5Cβ | 72 | 80 |
| 5Cα | 68 | 62 |
| 5Dε | 48 | 70 |
| 5Dδ | 57 | 96 |
| 5Dβ | 76 | 102 |
| 5Dα | 88 | 78 |
| 8Aε | 58 | 71 |
| 8Aδ | 72 | 89 |
| 8Aβ | 103 | 91 |
| 8Aα | 42 | 83 |
| 8Bε | 92 | 84 |
| 8Bδ | 90 | 112 |
| 8Bβ | 50 | 97 |
| 8Bα | 57 | 80 |
| 8Cε | 76 | 76 |
| 8Cδ | 73 | 72 |
| 8Cβ | 111 | 93 |
| 8Cα | 84 | 65 |
| 8Dε | 73 | 82 |
| 8Dδ | 81 | 63 |
| 8Dβ | 68 | 67 |
| 8Dα | 85 | 66 |
| 3Aεκ | | |
| 3Aδκ | 66 | 73 |
| 3Aβκ | 64 | 94 |
| 3Aακ | 64 | 87 |
| 3Bεκ | | |
| 3Bδκ | 59 | 93 |
| 3Bβκ | 61 | 95 |
| 3Bακ | 100 | 94 |
| 3Cεκ | | |
| 3Cδκ | 65 | 93 |
| 3Cβκ | 63 | 96 |
| 3Cακ | 71 | 99 |
| 3Dεκ | 73 | 88 |
| 3Dδκ | 81 | 97 |
| 3Dβκ | | |
| 3Dακ | 76 | 104 |
| 4Aεκ | | |
| 4Aδκ | 39 | 102 |
| 4Aβκ | 79 | 70 |
| 4Aακ | 43 | 76 |
| 4Bεκ | | |
| 4Bδκ | 56 | 96 |
| 4Bβκ | 82 | 79 |
| 4Bακ | 42 | 77 |
| 4Cεκ | | |
| 4Cδκ | 52 | 96 |
| 4Cβκ | 67 | 65 |
| 4Cακ | 52 | 90 |
| 4Dεκ | | |
| 4Dδκ | 50 | 83 |
| 4Dδκ | 64 | 97 |
| 4Dακ | 80 | 77 |
| 5Aεκ | | |
| 5Aδκ | 90 | 75 |
| 5Aβκ | 47 | 97 |
| 5Aακ | 91 | 103 |
| 5Bεκ | | |
| 5Bδκ | 46 | 78 |
| 5Bβκ | 63 | 100 |
| 5Bακ | 74 | 89 |
| 5Cεκ | | |
| 5Cδκ | 59 | 99 |
| 5Cβκ | 76 | 92 |
| 5Cακ | 93 | 109 |
| 5Dεκ | | |
| 5Dδκ | 40 | 92 |
| 5Dβκ | 64 | 86 |
| 5Dακ | 50 | 86 |
| 8Aεκ | 59 | 92 |
| 8Aδκ | 63 | 97 |
| 8Aβκ | 76 | 96 |
| 8Aακ | 53 | 77 |
| 8Bεκ | | |
| 8Bδκ | 80 | 95 |
| 8Bβκ | 44 | 81 |
| 8Bακ | 64 | 103 |
| 8Cεκ | 66 | 86 |
| 8Cδκ | 79 | 95 |
| 8Cβκ | 66 | 95 |
| 8Cακ | 31 | 92 |
| 8Dεκ | | |
| 8Dδκ | 65 | 90 |
| 8Dακ | 90 | 88 |
| 8Dακ | 51 | 84 |
| AG 1M | 89 | 73 |
| AG 2M | 80 | 47 |
| AG 3M | 123 | 63 |
| AG 4M | 100 | 74 |
| AG 5M | 87 | 68 |
| AG 6M | 76 | 57 |
| AG 7M | 99 | 63 |
| AG 8M | 69 | 87 |

HIV reverse transcriptase is multifunctional, possessing both DNA polymerase and RNase H activities. In separate, representative assays, mappicine analogs were tested for their ability to inhibit the RNA-dependent DNA polymerase activity of HIV reverse transcriptase and for their ability to inhibit the RNase H activity of HIV reverse transcriptase. The assay for RNA-dependent DNA polymerase activity is discussed in Borkow, G. et al., “Inhibition of the Ribonuclease H and DNA Polymerase Activities of HIV-1 Reverse Transcriptase by N-(4-tert-Butylbenzoyl)-2-hydroxy-1-naphthaldehyde Hydrazone,” Biochemistry 1997, 36, 3179-3185, a copy of which is attached hereto, and the disclosure of which is incorporated herein by reference. Assays were carried out in the absence and in the presence of mappicine analogs (10 μM final concentration). The results are reported as % residual activity, which is the RNA-dependent DNA polymerase activity of the enzyme in the presence of the mappicine analog divided by the RNA-dependent activity of the enzyme in the absence of the mappicine analog, multiplied by 100.

The assay for RNase H activity of HIV reverse transcriptase was the fluorescence-based assay described in detail in the Experimental section and discussed in U.S. Provisional Patent Application Serial No. 60/318,359. Assays were carried out in the absence and in the presence of mappicine analogs (10 μM final concentration). The results are reported as % residual activity, which is the RNase H activity of the enzyme in the presence of the mappicine analog divided by the RNase H activity of the enzyme in the absence of the mappicine analog, multiplied by 100. Alternatively, the results are reported as % inhibition, which is calculated as the ratio of the RNase H activity of the enzyme in the presence of the mappicine analog to the enzyme activity in the absence of the mappicine analog, multiplied by 100, and then subtracting this number from 100.

The results of biological testing of the mappicine analogs of the present invention were quite surprising. At a concentration of 10 μM, the mappicine analogs tested showed inhibitory activity against HIV reverse transcriptase via inhibitory activity against the RNA-dependent DNA polymerase activity and/or inhibitory activity against the RNase H activity of HIV reverse transcriptase. In general, mappicine analogs that very closely resembled the natural product were less active than more distant analogs, differing, for example, in at least two substituents. Some of these more distant analogs were, however, found to be quite active. While many mappicine analogs tested. surprisingly exhibited an appreciable level of RNase H inhibition at the concentration level of the studies, mappicine ketone analogs (illustrated in formula (2) above, and previously shown to be active against DNA viruses) were somewhat less active than other mappicine analogs (for example, mappicine alcohols, in which R¹ of formula (1) above is H).

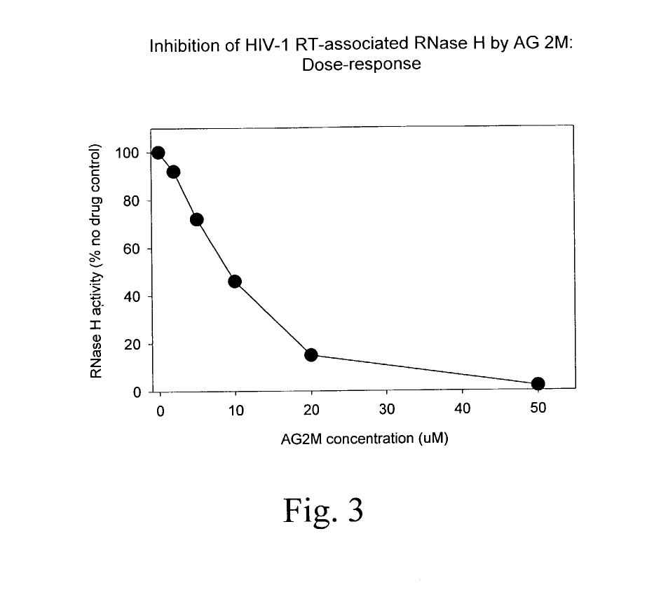

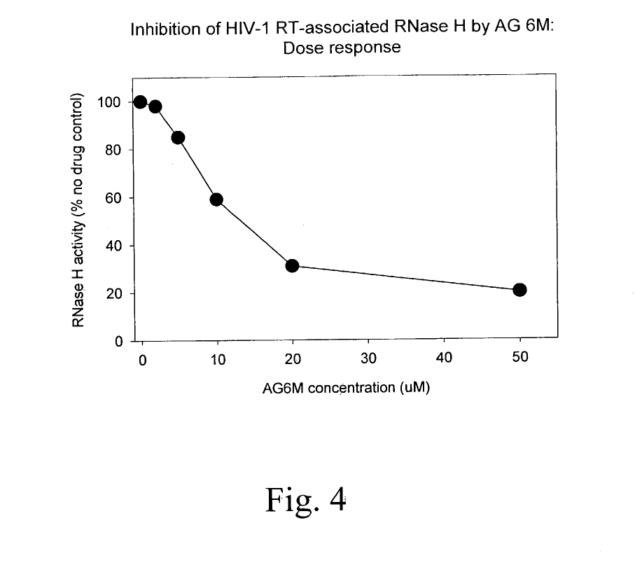

Two quite active mappicine analogs, AG 2M and AG 6M, are shown below. Dose-response curves for mappicine alcohols AG 2M and AG 6M are illustrated in FIGS. 2 and 3, respectively. Both of these mappicine possess inhibitory activity against HIV-1 RNase H (IC₅₀<10 μM; see Table 3) comparable to the known RNase H inhibitor BBNH. The results have been confirmed in cell culture viral growth assays (Table 3), thereby supporting the postulate that RNase H binding and anti-viral activity are linked.

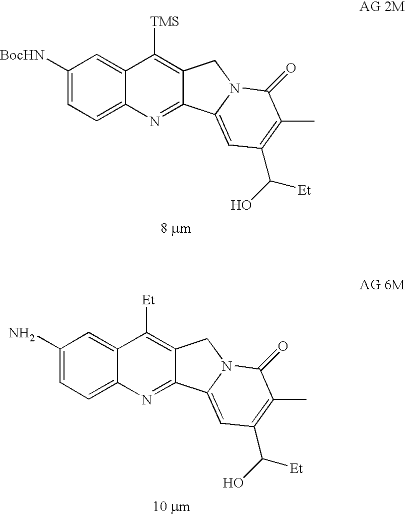

Based on the above results, mappicine analogs exhibit strong potential to provide extremely potent RNase H inhibitors. Such potent inhibitors are a welcome addition to the current arsenal for treatment of AIDs or other retroviral diseases, either alone or in combination with existing drugs.

TABLE 3

| | IC₅₀ (μM) against HIV RNase H | EC₅₀ (μM) against |
|---|---|---|
| COMPOUND | in vitro | HIV-1 replication |
| AG 2M | 8 | ≈5 |
| AG 6M | 10 | ≈5 |
| BBNH | 1.8 | 1.5 |

Other than the mappicine analogs described herein, there have been only three published reports of compounds able to inhibit HIV-1 reverse transcriptase associated RNase H activity with IC₅₀<10 μM. These compounds are N-4-(t-butylbenzoyl)-2-hydroxy-1-naphthaldehyde hydrazone (BBNH, see for example Borkow, G. et al., “Inhibition of the Ribonuclease H and DNA Polymerase Activities of HIV-1 Reverse Transcriptase by N-4-(tert-butylbenzoyl)-2-hydroxy-1-naphthaldehyde hydrazone,” Biochemistry 1997, 36, 3179-3185), 2-[(4-chlorophenyl)-hydrazono]-malonic acid (CPHM, see for example Gabbara, S. et al., “Inhibitors of DNA Strand Transfer Reactions Catalyzed by HIV-1 Reverse Transcriptase,” Biochemistry 1999, 38, 13070-13076), and 4-[5-(benzoylamino)thien-2-yl]-2,4-dioxobutanoic acid (BTOBA, see for example Shaw-Reid, C. et al., “Inhibition of HIV-1 ribonuclease H by a novel diketo acid, 4-[5-(benzoylamino)thien-2-yl]-2,4-dioxobutanoic acid,” Journal of Biological Chemistry 2003, 278, 2777-2780). Of these three, only BBNH has demonstrated antiviral activity against HIV-1 replication in cultured cells. However, BBNH is highly toxic to cells at concentrations only slightly above those that inhibit virus replication. Neither CPHM nor BTOBA are able to penetrate cells, and thus they cannot inhibit HIV-1 replication in cultured cells. Thus, BBNH, CPHM and BTOBA do not have therapeutic potential for the treatment of HIV-1 infection in humans. In contrast to BBNH, CPHM and BTOBA, the mappicine analogs described herein are capable of inhibiting HIV-1 replication in cultured cells and show little toxicity to cells. Thus, they provide the first example of an RNase H inhibitor with potential therapeutic utility.

Pharmaceutical Compositions

The present invention provides a broad variety of compositions prepared from compounds of the present invention. Such compositions have utility for human and veterinary antiviral use, and for treating viral infections in plants, e.g., agricultural or ornamental seeds and plants. Such compositions comprise a carrier which is acceptable for the intended end use together with at least one inventive compound. For example, in veterinary use, the carrier may be a liquid, or spray, or may be formulated in a solid, non-degradable or degradable form for insertion in the rumen. For agricultural use, the compound can be mixed with a fertilizer, other microbiocides such as fungicides, or insecticides and the like. The present compounds may also be formulated in powders or sprays for application to plant surfaces.

The pharmaceutical compositions of this invention comprise one or more compounds of the present invention in admixture with an inert pharmaceutically acceptable carrier or diluent. Compositions may contain an effective amount of the inventive compound in one unit, such as in a single pill, capsule, or pre-measured intravenous dose or pre-filled syringe for injection, or, as is frequently the case, the composition may be prepared in individual dose forms where one unit, such as a pill, contains a sub-optimal dose with the user being instructed to take two or more unit doses per treatment. When the composition is presented as a cream, it contains a discrete amount of drug and the user applies an effective amount of the cream one or more times until the disease is in remission or has been effectively treated. Concentrates for later dilution by the end user may also be prepared, for instance for IV formulations and multi-dose injectable formulations.

Carriers or diluents contemplated for use in these compositions are generally known in the pharmaceutical formulary arts. Reference to useful materials can be found in well known compilations such as Remington's Pharmaceutical Sciences, Mack Publishing Co., Easton, Pa., 18042, U.S.A.

The nature of the composition and the pharmaceutically acceptable carrier or diluent will, of course, depend upon the intended route of administration, for example, by intravenous and intramuscular injection, parenterally, topically, orally, or by inhalation.

For parenteral administration the pharmaceutical composition may be in the form of a sterile injectable liquid such as an ampule or an aqueous or nonaqueous liquid suspension.

For topical administration the pharmaceutical composition may be in the form of a cream, ointment, liniment, lotion, paste, spray or drops suitable for administration to the skin, eye, ear, nose or genitalia.

For oral administration the pharmaceutical composition may be in the form of a tablet, capsule, powder, pellet, atroche, lozenge, syrup, liquid, or emulsion.

The pharmaceutically acceptable carrier employed may be either a solid or liquid. Exemplary of solid carriers are lactose, kaolin, terra alba, sucrose, talc, gelatin, agar, pectin, acacia, magnesium stearate, mannitol, stearic acid and the like.

Examples of appropriate pharmaceutically acceptable liquid carriers or diluents include: for aqueous systems, water; for non-aqueous systems, ethanol, glycerin, propylene glycol, corn oil, cottonseed oil, peanut oil, sesame oil, liquid paraffins and mixtures thereof with water. For aerosol systems, pharmaceutically acceptable carriers include dichlorodifluoromethane, chlorotrifluoroethylene and compressed carbon dioxide. Also, in addition to the pharmaceutical carrier or diluent, the instant compositions may include other ingredients such as stabilizers, antioxidants, preservatives, lubricants, suspending agents, viscosity modifiers and the like, provided that the additional ingredients do not have a detrimental effect on the therapeutic action of the instant compositions. Similarly, the carrier or diluent may include time delay materials well known to the art, such as glyceryl monostearate or glyceryl distearate alone or with a wax, ethylcellulose, hydroxypropylmethylcellulose, methylmethacrylate and the like.

To obtain a stable water soluble dose form, a pharmaceutically acceptable salt of a compound of the present invention is dissolved in an aqueous solution of an organic or inorganic acid or base. If a soluble salt form is not available, the inventive compound may be dissolved in a suitable co-solvent or combinations thereof. Examples of such suitable cosolvents include, but are not limited to, alcohol, propylene glycol, polyethylene glycol 300, polysorbate 80, glycerin and the like in concentrations ranging from 0-60% of the total volume.

It will be appreciated that the actual preferred dosages of the compounds of the present invention used in the pharmaceutical and other compositions of this invention will vary according to the particular complex being used, the particular composition formulated, the mode of administration and the particular site, host and disease being treated. These compounds are active in the concentration ranges of two commercial antiviral drugs, Cytovene (ganciclovir) and Zovirax (acyclovir). For example, the latter is manufactured in 200 mg capsules with instructions for treating herpes simplex viruses by taking one capsule every 4 hours, but not to exceed 5 capsules per day.

Experimental

Assay for Ribonuclease H Activity Based on Fluorescence Resonance Energy Transfer (FRET)

Preparation of the Fluorophore-RNA/Quencher-DNA Hybrid Duplex Substrate.

The RNA/DNA hybrid duplex substrate for use in the assay comprises an RNA oligonucleotide of sequence 5′-GAU CUG AGC CUG GGA GCU-3′, modified at the 3′-end with Aminolink-2 and derivatized with fluorescein isothiocyanate, to provide a modified RNA oligonucleotide of the sequence 5′-GAU CUG AGC CUG GGA GCU-fluorescein-3′, annealed to a complementary DNA oligonucleotide of the sequence 5′-AGC TCC CAG GCT CAG ATC-3′ modified at the 5′-end with Aminolink-2 and derivatized with the FRET acceptor DABCYL succinimidyl ester, to provide a modified DNA oligonucleotide of the sequence 5′-DABCYL-AGC TCC CAG GCT CAG ATC-3′.

To prepare the 3′-fluorescein-RNA/5′-DABCYL-DNA hybrid duplex substrate, a known amount of 3′-fluorescein-RNA was dissolved in 20 mM Tris buffer (pH 8.0, 37° C.) to provide a final concentration of 5 μM. Two equivalents of the 5′-DABCYL-DNA oligonucleotide were added, and the mixture was heated to 90° C. for 5 min and cooled slowly to room temperature to allow duplex formation. The positioning of the fluorescein donor at the 3′-end of the RNA oligonucleotide and the DABCYL acceptor at the 5′-end of the DNA oligonucleotide provides a very close proximity of the donor and acceptor that results in a very intense quenching of the fluorescein emission in the intact RNA/DNA hybrid duplex substrate due to the spectral overlap of the fluorescence emission of fluorescein with the absorption spectrum of DABCYL. In addition, DABCYL is non-fluorescent, and thus cannot contribute any light emission. Both of these factors result in a very low background and provide a high signal-to-noise in the assay measurements. The ratio of the donor fluorescence in the absence and in the presence of its quencher is approximately fifteen-fold.

Microplate Assay Protocol for the Measurement of RNase H Activity Using the RNA/DNA Hybrid Duplex Substrate.

Reaction assay mixtures contained 5 μl of a stock solution of 2.5 μM RNA/DNA hybrid duplex substrate added to 85 μl of assay buffer (50 mM Tris, pH 8.0, 37° C., containing 60 mM KCl and 2.5 mM MgCl₂), prepared in the wells of a 96-well fluorescence microtiter plate, and warmed to 37° C. using the temperature control of the SpectraMax Gemini XS microplate spectrofluorometer (Molecular Devices). Reactions were started by the addition of 5 μl of a solution of recombinant HIV-1 reverse transcriptase (usually providing a final concentration of 2.5 nM of the p51/p66 RT heterodimer in the assay), and mixing using the automatic mixing function of the microplate spectrofluorometer. The increase in fluorescence signal resulting from the loss of FRET due to the enzymatic hydrolysis of the RNA strand was measured over suitable time intervals (ranging from 3 minutes to 60 minutes), at an excitation wavelength of 490 nm and an emission wavelength of 528 nm, using a cut-off filter of 515 nm. Data analysis and curve fitting were carried out using the appropriate transform functions of the software SigmaPlot 2000 (SPSS Inc.).

Synthesis

4-(1-Hydroxypropyl)-6-iodo-3-methyl-1-pent-2-ynyl-1H-pyridin-2-one (3a).

To a solution of 4-(1-hydroxypropyl)-6-iodo-3-methyl-1H-pyridin-2-one (3) (prepared as set forth in de Frutos, O.; Curran, D. P. J. Comb. Chem. 2000, 2, 639) (123 mg, 0.42 mmol) in DME (4.0 mL) and DMF (1.0 mL) at 0° C. was added portionwise NaH (20 mg, 0.505 mmol, 60% in mineral oil). After 15 min, LiBr (124 mg, 0.84 mmol) was added and the cooling bath removed. 2-Pentynyl bromide (73 mg, 0.84 mmol) was added 15 min later and the mixture was heated in the dark at 65° C. for 20 h. After cooling, the reaction was diluted with EtOAc, washed with brine, dried over Na₂SO₄ and evaporated. The residue was purified by flash chromatography (gradient CH₂Cl₂ to CH₂Cl₂/EtOAc 80:20) to yield 3a (99 mg, 66%) as a white foam: ¹H NMR (300 MHz, CDCl₃) δ0.91 (t, J=7.3 Hz, 3H), 1.1 (t, J=7.4 Hz, 3H), 1.53-1.67 (m, 2H), 1.89 (s, 3H), 2.16 (q, J=7.4 Hz, 2H), 3.49 (bs, 1H), 4.63 (t, J=6.5 Hz, 1H), 4.94 (d, J=16.8 Hz, 1H), 5.02 (d, J=16.8 Hz, 1H), 7.07 (s, 1H); ¹³C NMR (75 MHz, CDCl₃) δ10.09, 12.14, 12.59, 13.58, 29.89, 44.75, 70.37, 73.20, 86.75, 94.95, 118.64, 124.04, 153.29, 162.62; IR (film, NaCl, cm−1) 3398, 2977, 1629, 1519, 1177; LRMS (70 eV, EI) m/z (rel int %) 359 (M+), 344 (100), 254, 128, 93, 77, 67, 59; HRMS m/z calcd for C₁₄H₁₈NO₂I (M+) 359.0382, found 359.0393.

General Procedure for Cascade Radical Reaction.

To a solution of iodopyridone (15 mg) in benzene (0.5 mL) was added the corresponding isonitrile (3.0 equiv) and hexamethylditin (2 equiv). The mixture was irradiated at rt with a 275W GE sunlamp for 4 h and 30 min. The solvent was evaporated and the residue purified by flash chromatography.

2-tert-Butyloxycarbonylamino-7-(1-hydroxypropyl)-8-methyl-12-trimethylsilanyl-11H-indolizino[1,2-b]quinolin-9-one (AG-2M).

Treatment of 4-(1-hydroxypropyl)-6-iodo-3-methyl-1-[3-(trimethylsilanyl)prop-2-ynyl]-1H-pyridin-2-one (3b) (15.0 mg, 0.037 mmol) according to the cascade radical reaction general (see, de Frutos, O.; Curran, D. P. J. Comb. Chem. 2000, 2, 639) procedure afforded AG-2M (12.3 mg, 67%) as a pale brown solid, after purification of the crude residue by flash chromatography (gradient CH₂Cl₂ to CH₂Cl₂/acetone 1:1): ¹H NMR (300 MHz, CDCl₃) δ0.69 (s, 9H), 0.97 (t, J=7.3 Hz, 3H), 1.61 (s, 9H), 1.78-1.88 (m, 2H), 2.16 (s, 3H), 3.89 (bs, 1H), 4.87 (t, J=6.9 Hz, 1H), 5.09 (d, J=18.8 Hz, 1H), 6.73 (s, 1H), 7.19 (d, J=7.0 Hz, 1H), 7.68 (d, J=7.0 Hz, 1H), 8.29 (s, 1H); LRMS (70 eV, EI) m/z (rel int %) 493 (M+), 437 (100), 419, 404, 393, 378, 73; HRMS m/z calcd for C₂₇H₃₅N₃O₄Si (M+) 493.2397, found 493.2388.

2-Amino-7-(1-hydroxypropyl)-8-methyl-12-trimethylsilanyl-11H-indolizino[1,2-b]quinolin-9-one (AG-3M).

To a solution of AG-2M (9.8 mg, 0.02 mmol) in CH₂Cl₂ (0.5 mL) was added TFA (0.3 mL). The mixture was stirred at rt for 5 h and then concentrated under reduced pressure. The residue was purified by flash chromatography (gradient CH₂Cl₂ to CH₂Cl₂/acetone 1:1) to yield AG-3M (5.1 mg, 65%) as an orange solid: ¹H NMR (300 MHz, CD₃OD) δ0.63 (s, 9H), 1.02 (t, J=7.3 Hz, 3H), 1.71-1.77 (m, 2H), 2.13-2.20 (m, 3H), 5.28 (s, 2H), 7.29 (dd, J=2.1 and 8.9 Hz, 1H), 7.39 (d, J=2.1 Hz, 1H), 7.54 (s, 1H), 7.88 (d, J=8.9 Hz, 1H; ¹³C NMR (125 MHz, CDCl₃) δ1.86, 10.21, 12.15, 29.99, 51.87, 71.14, 99.32, 124.86, 126.51, 127.89, 128.88, 130.31, 131.29, 134.61, 142.14, 143.46, 147.00, 151.01, 154.32, 161.39; LRMS (70 eV, EI) m/z (rel int %) 393 (M+), 378, 364, 73, 57; HRMS m/z calcd. for C₂₂H₂₇N₃O₂Si (M+) 393.1872, found 393.1883.

2-tert-Butyloxycarbonylamino-12-ethyl-7-(1-hydroxypropyl)-8-methyl-11H-indolizino[1,2-b]quinolin-9-one (AG-5M).

Treatment of 3a (15.0 mg, 0.04 mmol) according to the cascade radical reaction general procedure afforded AG-5M (13.2 mg, 70%) as a brown solid, after purification of the crude residue by flash chromatography (gradient CH₂Cl₂ to CH₂Cl₂/acetone 1:1): ¹H NMR (300 MHz, CD₃OD) δ0.97 (t, J=7.3 Hz, 3H), 1.34 (t, J=7.5 Hz, 3H), 1.63 (s, 9H), 1.79-1.87 (m, 2H), 2.18 (s, 3H), 2.64 (s, 1H), 2.91-3.09 (m, 2H), 4.87 (t, J=6.9 Hz, 1H), 4.96 (d, J=18.5 Hz, 1H), 5.17 (d, J=18.5 Hz, 1H), 7.32 (d, J=9.0 Hz, 1H), 7.74 (d, J=9.1 Hz, 1H), 7.88 (s, 1H); LRMS (70 eV, EI) m/z (rel int %) 449 (M+), 393, 375, 358, 349 (100), 332, 320, 91, 57; HRMS m/z calcd. for C₂₆H₃₁N₃O₄ (M+) 449.2314, found 449.2312.

2-Amino-12-ethyl-7-(1-hydroxypropyl)-8-methyl-11H-indolizino[1,2-b]quinolin-9-one (AG-6M).

To a solution of AG-5M (10.4 mg, 0.023 mmol) in CH₂Cl₂ (0.5 mL) was added TFA (0.3 mL). The mixture was stirred at rt for 5 h and then concentrated under reduced pressure. The residue was purified by flash chromatography (gradient CH₂Cl₂ to CH₂Cl₂/acetone 1:1) to yield AG-6M (6.4 mg, 79%) as an orange solid: ¹H NMR (300 MHz, CD₃OD) δ1.02 (t, J=7.4 Hz, 3H), 1.35 (t, J=7.5 Hz, 3H), 1.71-1.89 (m, 2H), 2.20 (s, 3H), 3.11 (q, J=7.5 Hz, 2H), 5.14 (s, 2H), 7.21 (d, J=2.2 Hz, 1H), 7.30 (dd, J=2.2 and 9.1 Hz, 1H), 7.53 (s, 1H), 7.84 (d, J=9.1 Hz, 1H); ¹³C NMR (125 MHz, CD₃OD) δ10.58, 12.27, 13.9, 23.94, 31.36, 50.89, 72.23, 104.58, 124.77, 128.93, 130.29, 130.6, 143.6, 144.28, 145.21, 148.69, 156.91, 163.32; LRMS (70 eV, EI) m/z (rel int %) 349 (M+), 322, 101, 91, 81, 69, 57 (100); HRMS m/z calcd for C₂₁H₂₃N₃O₂ (M+) 349.1790, found 349.1800.

2-Acetoxy-12-ethyl-7-(1-hydroxypropyl)-8-methyl-11H-indolizino[1,2-b]quinolin-9-one (AG-7M).

Treatment of 3a (15.0 mg, 0.04 mmol) according to the cascade radical reaction general procedure afforded AG-7M (16.1 mg, 98%) as a pale brown solid, after purification of the crude residue by flash chromatography (gradient CH₂Cl₂ to CH₂Cl₂/acetone 1:1): ¹H NMR (300 MHz, CDCl₃) δ0.98 (t, J=7.4 Hz, 3H), 1.26 (t, J=7.6 Hz, 3H), 1.65-1.72 (m, 1H), 1.79-1.89 (m, 1H), 2.17 (s, 3H), 2.41 (s, 3H), 277 (q, J=7.6 Hz, 2H), 4.86-4.97 (m, 3H), 5.17 (d, J=18.6 Hz, 1H), 7.35-7.41 (m, 3H), 7.86 (d, J=8.9 Hz, 1H); LRMS (70 eV, EI) m/z (rel int %) 392 (M+), 350, 333, 167, 149 (100), 129, 99, 91, 71, 59; HRMS m/z calcd. for C₂₃H₂₄N₂O₃ (M+) 392.1736, found 392.1744.

12-Ethyl-2-hydroxy-7-(1-hydroxypropyl)-8-methyl-11H-indolizino[1,2-b]quinolin-9-one (AG-8M).

To a solution of AG-7M (13 mg, 0.033 mmol) in MeOH (0.4 miL) and H₂O (0.4 mL) was added K₂C0₃ (13.0 mg, 0.09 mmol). The mixture was stirred at rt for 2 h and then concentrated under reduced pressure. The residue was purified by flash chromatography (gradient CH₂Cl₂ to CH₂Cl₂/acetone 1:1) to yield AG-8M (9.1 mg, 78%) as a pale solid: ¹H NMR (300 MHz, CD₃OD) δ1.02 (t, J=7.3 Hz, 3H), 1.38 (t, J=7.5 Hz, 3H), 1.72-1.80 (m, 2H), 2.22 (s, 3H), 3.10-3.17 (m, 2H), 5.21 (s, 2H), 7.35-7.38 (m, 2H), 7.57 (s, 1H), 7.98 (d, J=9.8 Hz, 1H)); ¹³C NMR (125 MHz, CD₃OD) δ10.58, 12.26, 14.03, 22.5, 31.38, 72.24, 100.99, 106.19, 119.32, 123.76, 124.94, 128.85, 129.93, 131.91, 144.63, 145.17, 145.61, 150.82, 156.94, 158.46, 163.40; LRMS (70 eV, EI) m/z (rel int %) 350 (M+, 100), 333, 317, 292, 166, 69; HRMS m/z calcd for C₂₁H₂₂N₂O₃ (M+) 350.1630, found 350.1632.

Although the present invention has been described in detail in connection with the above examples, it is to be understood that such detail is solely for that purpose and that variations can be made by those skilled in the art without departing from the spirit of the invention except as it may be limited by the following claims.

What we claim is:

1. A method of inhibiting retroviral reverse transcriptase or hepadnaviral reverse transcriptase including the step of administering a pharmaceutically effective amount of a mappicine analog or a pharmaceutically acceptable salt thereof.

2. A method of treating a patient infected with a retrovirus or hepadnavirus including the step of administering a pharmaceutically effective amount of a mappicine analog or a pharmaceutically acceptable salt thereof.

3. The method of claim 2 wherein the retrovirus is human immunodeficiency virus.

4. The method of claim 2 wherein the hepadnavirus is human hepatitis B virus.

5 A method of treating a patient infected with a retrovirus including the step of administering a pharmaceutically effective amount of a compound have the following formula or a pharmaceutically acceptable salt thereof: 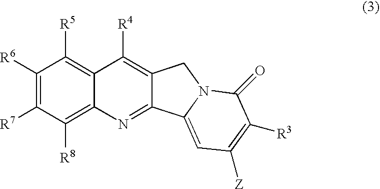
wherein Z is —CHOR¹R² or —C(O)R²;
R¹ is H, an alkyl group, an aryl group, —OC(O)OR^{a}, wherein R^{a} is an alkyl group, —C(O)R^{b} wherein R^{b} is an alkyl group, an aryl group, an alkoxy group, an amino group, an alkylamino group, a dialkylamino group, an aryl amino group, a diarylamino group, or an arylalkyl amino group;
R² is an alkyl group, an aryl group or an arylalkyl group;
R³ is H, an alkyl group, an hydroxyalkyl group or an aryl group;
R⁴-R⁸ are independently the same or different and are hydrogen, an alkyl group, an alkenyl group, an alkynyl group, an alkoxy group, an aryloxy group, an acyloxy group, a haloalkyl group, a perfluoroalkyl group, fluorine, chlorine, bromine, a carbamoyloxy group, a hydroxy group, a nitro group, a cyano group, a cyanoalkyl group, an azido group, an azidoalkyl group, a formyl group, a hydrazino group, a hydrazinoalkyl group, a hydroxyalkyl group, an alkoxyalkyl group, NR^{l}R^{m}, wherein R^{l} and R^{m} are independently hydrogen, an alkyl group, an aryl group, an arylalkyl group, or —C(O)R^{b}, an aminoalkyl group, an alkylaminoalkyl group, a dialkylaminoalkyl group, an aryl aminoalkyl group, a diarylaminoalkyl group, an arylalkyl aminoalkyl group;
—OC(O)OR^{a}, wherein R^{a} is an alkyl group,
—C(O)R^{b} wherein R^{b} is an alkyl group, an aryl group, an alkoxy group, an amino group, an alkylamino group, a dialkylamino group, an aryl amino group, a diarylamino group, an arylalkyl amino group
—SR^{c}, S(O)R^{c} or S(O₂)R^{c} wherein R^{c} is hydrogen, —C(O)R^{b}, an alkyl group, or an aryl group,
(CH₂)ₙSiR^{d}R^{e}R^{f} wherein n is an integer within the range of 0 through 10 and R^{d}, R^{e} and R^{f} are independently a C₁₋₁₀ alkyl group, a C₂₋₁₀ alkenyl group, a C₂₋₁₀ alkynyl group, an aryl group, a haloalkyl group, a cyanoalkyl group, an azidoalkyl group, a hydrazinoalkyl group, a hydroxyalkyl group, an alkoxyalkyl group, an aminoalkyl group, an alkylaminoalkyl group, a dialkylaminoalkyl group, an aryl aminoalkyl group, a diarylaminoalkyl group, an arylalkyl aminoalkyl group
or where R⁴ and R⁵, R⁵ and R⁶; R⁶ and R⁷; or R⁷ and R⁸ form together a chain of 3 or four groups selected from CH, CH₂, O, S, N, NH, N-alkyl or N-aryl.

6 The method of claim 5 wherein the patient is administered approximately 0.50-250 mg/kg of the compound or a pharmaceutically acceptable salt thereof.

7 The method of claim 5 wherein R¹ is H.

8 The method of claim 7 wherein neither R⁴ nor R6 is H.

9 The method of claim 7 wherein R⁶ is an alkyl group or (CH₂)ₙSiR^{d}R^{e}R^{f}, wherein R^{d}, R^{e} and R^{f} are independently a C₁₋₁₀ alkyl group, a C₂₋₁₀ alkenyl group, a C₂₋₁₀ alkynyl group, an aryl group, a haloalkyl group, a cyanoalkyl group, an azidoalkyl group, a hydrazinoalkyl group, a hydroxyalkyl group, an alkoxyalkyl group, an aminoalkyl group, an alkylaminoalkyl group, a dialkylaminoalkyl group, an aryl aminoalkyl group, a diarylaminoalkyl group, an arylalkyl aminoalkyl group.

10 The method of claim 7 wherein R⁶ is a hydroxyl group, an amino group or —NHBoc.

11 A method of inhibiting retroviral reverse transcriptase or hepadnaviral reverse transcriptase in a patient infected with a retrovirus including the step of administering a pharmaceutically effective amount of a compound have the following formula or a pharmaceutically acceptable salt thereof: 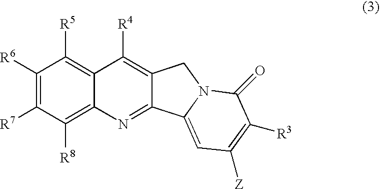
wherein Z is —CHOR¹R² or —C(O)R²;
R¹ is H, an alkyl group, an aryl group, —OC(O)OR^{a}, wherein R^{a} is an alkyl group, —C(O)R^{b} wherein R^{b} is an alkyl group, an aryl group, an alkoxy group, an amino group, an alkylamino group, a dialkylamino group, an aryl amino group, a diarylamino group, or an arylalkyl amino group;
R² is an alkyl group, an aryl group or an arylalkyl group;
R³ is H, an alkyl group, an hydroxyalkyl group or an aryl group;
R⁴-R⁸ are independently the same or different and are hydrogen, an alkyl group, an alkenyl group, an alkynyl group, an alkoxy group, an aryloxy group, an acyloxy group, a haloalkyl group, a perfluoroalkyl group, fluorine, chlorine, bromine, a carbamoyloxy group, a hydroxy group, a nitro group, a cyano group, a cyanoalkyl group, an azido group, an azidoalkyl group, a formyl group, a hydrazino group, a hydrazinoalkyl group, a hydroxyalkyl group, an alkoxyalkyl group, NR^{l}R^{m}, wherein R^{l} and R^{m} are independently hydrogen, an alkyl group, an aryl group, an arylalkyl group, or —C(O)R^{b}, an aminoalkyl group, an alkylaminoalkyl group, a dialkylaminoalkyl group, an aryl aminoalkyl group, a diarylaminoalkyl group, an arylalkyl aminoalkyl group
—OC(O)OR^{a}, wherein R^{a} is an alkyl group,
—C(O)R^{b} wherein R^{b} is an alkyl group, an aryl group, an alkoxy group, an amino group, an alkylamino group, a dialkylamino group, an aryl amino group, a diarylamino group, an arylalkyl amino group
—SR^{c}, S(O)R^{c} or S(O₂)R^{c} wherein R^{c} is hydrogen, —C(O)R^{b}, an alkyl group, or an aryl group,
(CH₂)ₙSiR^{d}R^{e}R^{f} wherein n is an integer within the range of 0 through 10 and R^{d}, R^{e} an R^{f} are independently a C₁₋₁₀ alkyl group, a C₂₋₁₀ alkenyl group, a C₂₋₁₀ alkynyl group, an aryl group, a haloalkyl group, a cyanoalkyl group, an azidoalkyl group, a hydrazinoalkyl group, a hydroxyalkyl group, an alkoxyalkyl group, an aminoalkyl group, an alkkylaminoalkyl group, a dialkylaminoalkyl group, an aryl aminoalkyl group, a diarylaminoalkyl group, an arylalkyl aminoalkyl group
or where R⁴ and R⁵, R⁵ and R⁶; R⁶ and R⁷; or R⁷ and R⁸ form together a chain of 3 or four groups selected from CH, CH₂, O, S, N, NH, N-alkyl or N-aryl.

12. The method of claim 11 wherein the patient is administered approximately 0.50-250 mg/kg of the compound or a pharmaceutically acceptable salt thereof.
